(19) United States
(12) Patent Application Publication
(10) Pub. No.: US 2006/0003961 A1
(43) Pub. Date: Jan. 5, 2006

(54) Negative regulation of hypoxia inducible factor 1 by OS-9

(75) Inventor: Gregg L. Semenza, Reisterstown, MD (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: THE JOHN HOPKINS UNIVERSITY, Baltimore, MD (US)

(21) Appl. No.: 11/156,163

(22) Filed: Jun. 17, 2005

Related U.S. Application Data
(60) Provisional application No. 60/581,208, filed on Jun. 18, 2004.

(51) Int. Cl. A61K 48/00; A61K 39/395; C12N 15/86

(52) U.S. Cl. 514/44; 435/456; 514/2; 424/143.1

(57) ABSTRACT

The present invention discloses that OS-9 interacts with both HIF-1a and HIF-1a prolyl hydroxylases. Overexpression of OS-9 promotes the hydroxylation of HIF-1a, HIF-1a binding to VHL, proteasomal degradation of HIF-1a, and loss of HIF-1-mediated transcription. OS-9 loss-of-function increases HIF-1a protein levels and HIF-1-mediated transcription under non-hypoxic conditions. These data indicate that OS-9 is an essential component of a multiprotein complex that regulates HIF-1a protein levels in an O₂ dependent manner. Agents which modulate this complex, and methods to identify such agents, are disclosed.

### RELATED APPLICATION DATA

This application claims priority from U.S. Provisional Application Ser. No. 60/581,208 filed Jun. 18, 2004, the entire contents of which is incorporated herein by reference.

### BACKGROUND OF THE INVENTION

1. Field of Invention

The invention relates generally to modulation of hypoxic effects and, more specifically, to methods of regulation of HIF-1 dependent O₂ homeostasis by exploitation of OS-9/HIF-1α and OS-9/HIF-1α prolyl hydroxylase interactions, including methods to identify agents as drug candidates that modulate these interactions.

2. Background Information

The ability of cells to sense and respond to changes in O₂ concentration is a universal property of all prokaryotic and eukaryotic species. In metazoan species, hypoxia-inducible factor 1 (HIF-1) functions as a master regulator of O₂ homeostasis by playing critical roles in both embryonic development and postnatal physiology. HIF-1 is a transcription factor that activates gene expression in response to hypoxia. One group of HIF-1 target genes encode proteins that enable cells to survive O₂ deprivation by providing an O₂-independent means of ATP production (glucose transporters and glycolytic enzymes) or by inhibiting hypoxia-induced apoptosis (growth/survival factors such as insulin-like growth factor 2). Another group of target genes encode proteins that increase tissue O₂ delivery by stimulating angiogenesis (e.g. vascular endothelial growth factor) or erythropoiesis (e.g. erythropoietin).

HIF-1 is a heterodimer consisting of an O₂-regulated HIF-1α subunit and a constitutively-expressed HIF-1β subunit. The amino-terminal half of each subunit consists of basic helix-loop-helix (bHLH) and PAS domains that mediate heterodimerization and DNA binding. The carboxyl terminal half of HIF-1α contains the domains that regulate its half-life and transcriptional activity in an O₂-dependent manner.

The molecular mechanism by which changes in O₂ concentration are transduced to changes in gene expression mediated by HIF-1 has been elucidated. The O₂ dependent degradation of HIF-1α involves ubiquitination and degradation by the 26S proteasome. The von Hippel-Lindau tumor suppressor protein (VHL) is required for this process and renal carcinoma cells lacking functional VHL constitutively express HIF-1α and HIF-1 target genes under non-hypoxic conditions. VHL forms a complex with elongin B, elongin C, cullin 2, and RBX1 to form an E3 ubiquitin-protein ligase capable of functioning with E1 ubiquitin-activating and E2 ubiquitin-conjugating enzymes to mediate ubiquitination of HIF-1α.

VHL binds to HIF-1α only when the latter has been modified by hydroxylation at proline residue 402 and/or 564. Three prolyl hydroxylases were identified in mammalian cells and shown to utilize O₂ as a substrate to generate 4-hydroxyproline at residue 402 and/or 564 of HIF-1α. The hydroxylation reaction also requires 2-oxoglutarate (α-ketoglutarate) as a substrate and generates succinate as a side product. The mammalian HIF-1α prolyl hydroxylases are homologues of EGL-9, which was identified as the HIF-1α prolyl hydroxylase in C. elegans by genetic studies. Alternative designations for the three mammalian homologues include EGLN (EGL Nine homologue), PHD (Prolyl Hydroxylase Domain protein), and HPH (HIF-1α Prolyl Hydroxylase) 1, 2, and 3. The HIF-1α prolyl hydroxylases have a relatively high Kₘ for O₂ that is slightly above its atmospheric concentration, such that O₂ is rate limiting for enzymatic activity under physiological conditions. As a result, changes in the cellular O₂ concentration are directly transduced into changes in the rate at which HIF-1α is hydroxylated, ubiquitinated, and degraded.

HIF-1α transactivation domain function is also regulated by O₂-dependent hydroxylation of asparagine residue 803, which blocks the binding of the coactivators CBP and p300. FIH-1 (factor inhibiting HIF-1), which was identified in a yeast two-hybrid screen as a protein that interacts with and inhibits the activity of the HIF-1α transactivation domain, functions as the asparaginyl hydroxylase. As in the case of the prolyl hydroxylases, FIH-1 appears to utilize O₂ and 2-oxoglutarate and contain Fe (II) in its active site, although it has a Kₘ for O₂ that is three times lower than the prolyl hydroxylases.

One remarkable aspect of the O₂ sensing system described above is its plasticity. Expression levels of the PHDs vary from one cell type to another and in response to various physiological stimuli, including hypoxia. Thus, the O₂ dose-response curve may be shifted to the left or right under different developmental or physiological conditions. Alternative splicing of the primary RNA transcripts for two of the PHDs provides another potential mechanism for modulating prolyl hydroxylase activity.

OS-9, a protein which remains relatively uncharacterized, is ubiquitously present in human tissues, as shown by mRNA distribution. The protein is over-expressed in certain sarcomas, however a function had not been assigned to the protein with any certainty. While it has been shown that OS-9, for example, interacts with meprin β, and may be involved in ER-to-Golgi transport, the essential function of OS-9 remains open.

### SUMMARY OF THE INVENTION

To understand how cells respond to altered oxygenation, an experimental paradigm was used to manipulate known components of oxygen responsive proteins. The present invention discloses that the protein OS-9 interacts with both HIF-1α and PHDs. The formation of this ternary complex promotes PHD-mediated hydroxylation of HIF-1α, binding of VHL, and proteasomal degradation of HIF-1α.

In one embodiment, a method of modulating hypoxia-inducible factor 1 (HIF-1) activity is envisaged including contacting a OS-9 and HIF-1 or a fragment thereof in a sample, with an agent that modulates OS-9 activity or expression and determining the effect of the contacting on the activity of HIF-1 or fragment thereof, where modulation of OS-9 activity or expression affects HIF-1 activity.

In a related aspect, the modulating agent inhibits the activity, synthesis, or stability of OS-9, resulting in increased HIF-1 activity or the modulating agent stimulates the activity, synthesis, or stability of OS-9, resulting in decreased HIF-1 activity.

Such agents are envisaged to include, but are not limited to, an antibody, protein, small molecule, or a nucleic acid. Further, the nucleic acid may be an aptamer, antisense RNA, or gene silencing RNA, where the gene silencing RNA includes, but is not limited to, a dsRNA, siRNA, stRNA, or RNA silencing hairpin.

In a related aspect, the protein is an exogenous OS-9 isoform, where the isoforn exhibits activity antagonistic to the OS-9 endogenous to the sample. Further, the sample includes, but is not limited to, a cell, tissue, or organ transfected with an expression vector comprising an operably linked DNA encoding the exogenous isoform.

In another related aspect, OS-9 activity may be increased by overexpression of an endogenous isoform of OS-9.

In another related aspect, increased HIF-1 activity stimulates angiogenesis, glucose metabolism, or cell survival. Alternatively, decreased HIF-1 activity inhibits angiogenesis, glucose metabolism, or cell survival. Further, the determining step may include, but is not limited to, analysis of OS-9 protein levels.

In another related aspect, OS-9 modulation affects interaction between OS-9 and HIF-1 and/or OS-9 and a prolyl hydroxylase (PHD), where such interaction may be determined by methods including, but not limited to, fluorescence resonance energy transfer (FRET), two-hybrid assay, mass spectrometry, protein chip assay, SOS recruitment assay, and RNA polymerase III based two-hybrid assay.

In one aspect, HIF-1 activity corresponds to HIF-1 protein stability and/or transactivation of O₂/hypoxia dependent gene expression via HIF-1, where transactivation of O₂/hypoxia dependent gene expression can be monitored by determining expression of a gene, gene-fusion construct, or gene fragment, which gene, gene-fusion construct, or gene fragment expression is regulated by a hypoxia response element (HRE).

In a related aspect, the sample includes an HRE-containing expression vector, which expression from the vector is responsive to 0₂/hypoxia dependent transactivation, where the vector expresses a reporter protein. Further, the reporter may lead to the production of a protein that can be detected by virtue of its fluorescent, luminescent, enzymatic, or immunologic properties. Further, such reporter proteins may include, but are not limited to, luciferase, green fluorescent protein, chloramphenicol acetyltransferase (CAT), β-galactosidase (β-Gal), and alkaline phosphatase.

In another related aspect, the vector expresses a fusion protein comprising HIF-1α, or a fragment thereof, and a gene reporter. Moreover, the gene reporter includes, but is not limited to, GFP or luciferase.

In one aspect, HIF-1 protein stability can be monitored by determining interaction between HIF-1, an HIF-1 subunit, or an HIF-1 fragment and a PHD or PHD fragment, and/or a von Hippel-Lindau tumor suppressor protein (VHL), or VHL fragment. Further, HIF-1 can be monitored by determining interaction between HIF-1, an HIF-1 subunit or HIF-1 fragment and FIH -1, where the HIF-1 subunit is HIF-1α. In a further related aspect, the PHD is PHD1, PHD2, or PHD3.

Further, protein stability can be monitored by determining ubiquitylation of HIF-1, HIF-1α, or fragment thereof, where ubiquitylation results in degradation of HIF-1, HIF-1α, or fragment thereof by a proteasome. Moreover, OS-9 dependent affects on HIF-1 protein stability and/or transactivation of O₂/hypoxia dependent gene expression effects modulation of glucose transporter expression, glycolytic enzyme expression, or growth/survival factor expression.

In another embodiment, a method of identifying an OS-9 modulating agent is envisaged including contacting OS-9 and HIF-1, an HIF-1 subunit, or a fragment thereof, with a test agent in a sample, allowing interaction between the agent-contacted OS-9 and HIF-1, HIF-1 subunit, or a fragment thereof, and determining HIF-1 activity, where the test agent inhibits the activity, synthesis, or stability of OS-9, resulting in increased HIF-1 activity or the test agent stimulates the activity, synthesis, or stability of OS-9, resulting in decreased HIF-1 activity.

In a related aspect, where the sample is a cell, tissue, or organ, the level of OS-9 protein can be determined subsequent to contact with the test agent.

In a further related aspect, an agent identified by the method is envisaged, where the agent may be an RNA. Further, the RNA sequence is encoded by a nucleic acid comprising gtacaaacagcgctatgag (SEQ ID NO:1). Further, there is a protein sequence in the disclosure documents that is not a figure, this will need a sequence identifier and must be listed in the sequence listing. Moreover, a pharmaceutical composition comprising a pharmaceutically acceptable carrier and a nucleic acid comprising SEQ ID NO:1 is also envisaged.

In one aspect, such an agent includes, but is not limited to, a small molecule, mineral, protein, peptide, hormone, nucleic acid, lipid, carbohydrate, vitamin, or co-enzyme. Further, the method includes determining HIF-1α protein levels, wherein the sample is a cell, tissue, or organ.

In another aspect, the sample comprises an expression vector encoding a gene, gene-fusion construct, or gene fragment, where expression from the vector is responsive to O₂/hypoxia dependent transactivation. Further, vectors comprising reporter proteins are envisaged to include a gene-fusion construct regulated by a hypoxia response element (HRE). Further, such constructs include at least one HIF-1/OS-9 binding site.

In one embodiment, a method of modulating a regulator of O₂ homeostasis in a subject including altering the expression, stability, or activity of OS-9 is envisaged, where the regulator is hypoxia inducible factor 1 (HIF-1). Further, the method may include administering to the subject or contacting the subject with an agent which modulates OS-9 expression, stability, or activity, where the modulating agent is a small molecule, nucleic acid, or protein. Moreover, the agent inhibits the synthesis or stability of OS-9 protein or mRNA or the agent inhibits the interaction between OS-9 and HIF-1, HIF-1 subunit or fragment thereof, or the interaction between OS-9 and PHDs. Alternatively, the agent stimulates the synthesis or stability of OS-9 protein or mRNA or the agent stimulates the interaction between OS-9 and HIF-1, HIF-1 subunit or fragment thereof, or the interaction between OS-9 and PHDs.

In a related aspect, the agent inhibits the activity, synthesis, or stability of OS-9, resulting in increased HIF-1 activity or the agent stimulates the activity, synthesis, or stability of OS-9, resulting in decreased HIF-1 activity.

In another aspect, the subject demonstrates an ischemic condition, to include, but is not limited to, a coronary, cerebral, or vascular disorder. In a related aspect, the subject demonstrates a cell proliferating disorder, where the disorder is cancer.

In one aspect, increased HIF-1 activity stimulates angiogenesis, glucose metabolism, or cell survival. In a related aspect, decreased HIF-1 activity inhibits angiogenesis, glucose metabolism, or cell survival.

In another aspect, the agent is an OS-9 isoform, a small molecular weight compound or a vehicle encoding OS-9 or an OS-9 isoform, where the vehicle is a plasmid or viral vector.

In another embodiment, a method of treatment including administering to a subject a pharmaceutically acceptable carrier and an OS-9 modulating agent, where the agent alters the expression, stability, or activity of OS-9. In a related aspect, the agent inhibits the activity, synthesis, or stability of OS-9, resulting in increased hypoxia inducible factor 1 (HIF-1) activity or the agent stimulates the activity, synthesis, or stability of OS-9, resulting in decreased HIF-1 activity.

In a further related aspect, the subject presents an ischemic condition and/or a cell proliferating disorder, including but not limited to, coronary, cerebral, or vascular disorders, and cancer.

Exemplary methods and compositions according to this invention, are described in greater detail below.

### BRIEF DESCRIPTION OF THE DRAWINGS

FIG. 1 illustrates yeast two-hybrid screening vectors for identifying OS-9 cDNA. (A) For two-hybrid screening, the bait vector encoded a chimeric protein consisting of the DNA-binding domain from the yeast GAL4 transcription factor (GAL4 DBD) fused to residues 576-826 of HIF-1α. The prey vectors encoded the GAL4 transactivation domain (GAL4 TAD) fused to residues encoded by human brain cDNAs. (B) Identification of a conserved domain in OS-9. Conserved Domain Database search using the human OS-9 sequence revealed animal, plant, and yeast proteins (SEQ ID NO'S 5-14) with significant similarity to OS-9 including a highly conserved 18-amino-acid sequence. B, hydrophobic residue (F, I, L, or V).

FIG. 2 demonstrates the interaction between OS-9 and HIF-1α. (A) GST fusion proteins containing the indicated HIF-1α residues were incubated with ³⁵S-labelled in vitro-translated HA-OS-9, captured on glutathione (GSH)-Sepharose beads and analyzed by SDS-PAGE and autoradiography. T/I, 10% total input of HA-OS-9. (B) 293 cells were transfected with HIF-1α or HA-OS-9 expression vector. Immunoblot assays were performed with antibodies against HIF-1α or HA using whole cell lysate (WCL) directly or after immunoprecipitation (IP) with anti-HA affinity matrix.

FIG. 3 shows the effect of OS-9 on HIF-1α protein levels and HIF-1 transcriptional activity. (A) Hep3B cells were co-transfected with control reporter pSV-Renilla, HIF-1-dependent firefly luciferase reporter p2.1, and the indicated amount (in ng) of expression vector encoding HA-OS-9, HIF-1α, or empty vector (EV). Cells were exposed to 20% (open bars) or 1% (closed bars) O₂ for 16 h and the ratio of firefly:Renilla luciferase activity was determined. The results were normalized to those from cells transfected with EV and exposed to 20% O₂ (luciferase activity). The mean and standard deviation based on three independent transfections are shown. (B) Hep3B cells were co-transfected with pSV-Renilla, firefly luciferase reporter pG5E1bLuc (containing five GAL4 binding sites), expression vector encoding the GAL4 DNA-binding domain alone (Gal 0) or fused to HIF-1α residues 531-826 (Gal A), and empty vector or vector encoding HA-OS-9 (indicated amounts of plasmid DNA in ng). (C) 293 cells were co-transfected with empty vector or plasmid encoding HA-OS-9 with or without HIF-1α expression vector. Cell lysates were subjected to immunoblot assay using either an anti-HIF-1α or anti-HA monoclonal antibody.

FIG. 4 shows that OS-9 regulation of HIF-1α is dependent on prolyl hydroxylation and proteasome activity. (A) 293 cells were co-transfected with empty vector or HA-OS-9 expression vector and expression vector encoding wild type HIF-1α or the HIF-1α triple mutant (TM) P402A/P564A/N803A. Transfected cells were treated with vehicle or MG132 (10 μM) for 4 h. Cell lysates were subjected to immunoblot assay to detect HIF-1α or HA-OS-9. (B) 293 cells were co-transfected with: pSV-Renilla; HIF-1-dependent firefly luciferase reporter p2.1; empty vector (EV) or expression vector encoding HIF-1α or HIF-hTM; and HA-OS-9 or PHD2 expression vector. After 24 h, cells were lysed and the ratio of firefly:Renilla luciferase activity was determined. The results were normalized to those from cells transfected with EV (luciferase activity). The mean and standard deviation based on three independent transfections are shown. *P <0.05 for HIF-1α/PHD2- or HIF-1α/OS-9- compared to HIF-1α/EV-transfected cells.

FIG. 5 demonstrates the interaction between OS-9 and PHDs in human cells. 293 cells were transfected with the indicated expression vectors. Aliquots of whole cell lysate were used for immunoprecipitation (IP) assay using anti-HA affinity matrix followed by immunoblot (IB) assay with specific antibodies that recognize each PHD isoform (top panels) and HA (bottom panels). NS, non-specific cross-reacting protein.

FIG. 6 shows the effect of OS-9 on PHD2-mediated HIF-1α destabilization. (A) 293 cells were co-transfected with the indicated combination of FLAG-HIF-1α, HA-OS-9, and PHD2 expression vector. Aliquots of whole cell lysate were analyzed by immunoblot assay with antibodies that recognize FLAG or PHD2. FLAG-HIF-1α protein levels were quantified by densitometric analysis (band intensity). (B) 293 cells were co-transfected with pSV-Renilla, HIF-1-dependent firefly luciferase reporter p2.1, and HIF-1α, HA-OS-9, PHD2 or empty (EV) expression vector. After 24 h, cells were lysed and the ratio of firefly:Renilla luciferase activity was determined. The results were normalized to those from cells transfected with EV (luciferase activity). The mean and standard deviation based on three independent transfections are shown. *, significant effect of OS-9/HIF-1α co-transfection compared to HIF-1α alone (P<0.05). #, significant effect of HIF-1α/PHD2/OS-9 co-transfection compared to HIF-1α/PHD2 (P<0.05). (C) GST-HIF-1α (531-826) was incubated with cell lysate from PHD2-transfected 293 cells. After 10 min incubation at 30° C., the indicated amounts of in vitro-translated VHL and OS-9 were added and GST-HIF-1α was pulled down with glutathione-Sepharose. The presence of OS-9 and VHL was determined by SDS-PAGE and autoradiography.

FIG. 7 shows the effect of OS-9 on VHL binding, PHD2-mediated hydroxylation of HIF-1α, and binding of PHD2 to HIF-1α. 293 cells were transfected with empty (EV), PHD2 or HA-OS-9 expression vector and lysates were incubated with GST-HIF-1α (531-826) for 10 min at 30° C. (A) Aliquots of reaction mixtures were subjected to in vitro VHL binding assay (top panel; band intensity quantified by densitometry) or to immunoblot analysis using either an anti-hydroxyproline-564 (Hyp-564) or anti-GST monoclonal antibody (middle and bottom panels, respectively). (B) Aliquots of reaction mixtures were subjected to pull down assay. GST-HIF-1α (531-826) was captured on glutathione-Sepharose beads and analyzed by immunoblot using antibodies against PHD2 (top panel) or GST (bottom panel). (C) Aliquots of cell lysates were subjected to immunoblot analysis using either an anti-HA or anti-PHD2 antibody.

FIG. 8 demonstrates the down-regulation of OS-9 by RNA interference. 293 cells were transfected with empty (EV), scrambled negative control (SNC) short hairpin RNA (shRNA), or OS-9 shRNA expression vector. After 24 h, total RNA was isolated and used for cDNA synthesis. (A) Expression of GFP in transfected cells was determined by fluorescence microscopy. (B) cDNA was analyzed by PCR using primers specific for OS-9, HIF-1α and β-actin. (C) Expression of OS-9 mRNA and 18S rRNA was analyzed by real-time RT-PCR. The mean and standard deviation based on three independent PCR reactions are shown.*, P<0.05.

FIG. 9 shows the effect of OS-9 down-regulation on HIF-1α protein levels and HIF-1 activity. (A) 293 cells were transfected with the indicated amount of empty and shRNA expression vectors. Aliquots of whole cell lysate were analyzed by immunoblot assay with antibodies that recognize HIF-1α or HIF-1β. (B) 293 cells were co-transfected with pSV-Renilla, firefly luciferase reporter p2.1, and indicated amount of EV and shRNA expression vectors. After 24 h, cells were lysed and the ratio of firefly:Renilla luciferase activity was determined. The results were normalized to those cells transfected with EV (luciferase activity). The mean and standard deviation based on three independent transfections are shown. *P<0.05 compared with cells transfected with expression vector (15 μg) encoding shRNA_{SNC}

FIG. 10 illustrates negative regulation of HIF-1α protein stability and transcriptional activity under non-hypoxic conditions mediated by a multiprotein complex. Protein-protein interactions are indicated by solid double arrows and enzymatic activity is indicated by open or dotted arrows. B, elongin B; C, elongin C; Cu12, cullin 2; E2, ubiquitin-conjugating enzyme.

FIG. 11 is the amino acid sequence of human OS-9.

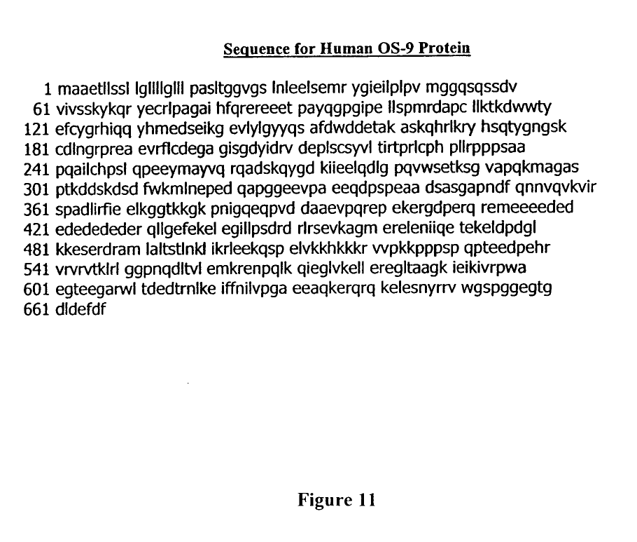

### DETAILED DESCRIPTION OF THE INVENTION

Before the present compositions and methods are described, it is understood that this invention is not limited to the particular methodology, protocols, and reagents described as these may vary. It is also to be understood that the terminology used herein is for the purpose of describing particular embodiments only, and is not intended to limit the scope of the present invention which will be described by the appended claims.

It must be noted that as used herein and in the appended claims, the singular forms “a”, “an”, and “the” include plural reference unless the context clearly dictates otherwise. Thus, for example, reference to “a cell” includes a plurality of such cells, reference to “a protein” includes one or more proteins and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the methods, devices, and materials are now described. All publications mentioned herein are incorporated herein by reference for the purpose of describing and disclosing the proteins, compounds, and methodologies which are reported in the publications which might be used in connection with the invention. Nothing herein is to be construed as an admission that the invention is not entitled to antedate such disclosure by virtue of prior invention.

As used herein, “modulating,” including grammatical variations thereof, means an adjustment or regulation of the degree or activity of a molecular entity. For example, a ligand that increases or decreases the activity or binding properties of a protein would be modulating the activity or binding of that protein. In a related aspect, such modulation can be negative (e.g., decreases the activty or binding) or positive (e.g., increases the activity or binding).

In a related aspect, OS-9 activity includes, but is not limited to, binding to and/or modulating the transactivation of HIF-1 and binding to and/or modulating prolyl hydroyxlase (PHD) activity.

O₂/hypoxia dependent gene expression includes, but is not limited to, expression of those genes whose modulation serves to mount the appropriate biological response(s) to changes in oxygen concentration. For example, genes that encode proteins that increase tissue O₂ delivery by stimulating angiogenesis (e.g. vascular endothelial growth factor) or erythropoiesis (e.g. erythropoietin).

As used herein, “transactivation,” including grammatical variations thereof, means gene activation via recognition of a regulatory element by a transcriptional factor (e.g., HIF-1).

As used herein “hypoxia response element,” including grammatical variations thereof, means a nucleic acid sequence found in many promoter regions whose genes are transactivated by HIF-1. For example, such promoters include, but are not limited to, elements identified in the promoters of genes encoding plasminogen activator inhibitor I, 6-phosphofructo-2-kinase, enolase 1, vascular endothelial growth factor, and erythropoietin.

As used herein “luminescent,” including grammatical variations thereof, means molecules (including those of biological origin) or moieties which use chemical energy to produce light.

As used herein “gene silencing RNA” means ribonucleic acid (RNA) sequences which “knock-down” the expression of genes, where such RNA sequences are homologous to a target mRNA and serve as a component of binding complexes which lead to target cleavage by enzymes such as Dicer-RDE-1 (see, e.g., McManus and Sharp, Nature Reviews (2002) 3:737-747). In a related aspect, such gene silencing RNA includes, but is not limited to, double stranded (ds) RNA, short interfering (si) RNA, small temporal (st) RNA, and RNA silencing hairpins. In another related aspect, such a gene silencing RNA is encoded in SEQ ID NO:1.

As used herein “physiological end-point” means the sum of a particular organic/biological process of an organism or of its parts or of a particular biological process.

As used herein “hypoxic environment” means surroundings where there is a deficiency of oxygen affecting a tissue, organ, cell, or organism or its parts.

As used herein “regulator of O₂ homeostasis” means a biological molecule (or molecules) which work in concert to provide an appropriate organic response to changes in O₂ tension. For example, HIF-1 and PHD are regulators of O₂ homeostasis.

As used herein “agent contacted,” including grammatical variations thereof, means a molecular moiety that has been exposed to an chemical entity, which contrasts from a naïve form of the same molecular moiety which has not been so exposed.

As used herein “ubiquitylation,” including grammatical variations thereof, means a ATP dependent reaction between unbiquitin and proteins that leads to the formation of a multiprotein complex and subsequent degradation of the ubiqutinated protein.

In the present invention OS-9 is demonstrated to be a negative regulator of HIF-1 that promotes prolyl hydroxylation by interacting with both HIF-1α and PHDs. Previously published data on OS-9 provided no clue that the protein was involved in the regulation of HIF-1 and O₂ homeostasis. OS-9 has been shown to interact with the proteins meprin-β and N-copine, has been localized primarily to endoplasmic reticulum membranes, and has been implicated to ER-to-Golgi transport of proteins in mammalian and yeast cells (Friedmann et al. 2002; Litovchick et al. 2002; Nakayama et al. 1999). Bioinformatic analyses indicate that OS-9 defines a family of proteins present in animals, plants, and yeast sharing extended sequence similarity, which suggests that they may share a common biochemical activity, the nature of which remains to be determined.

OS-9 is overexpressed in osteosarcomas, as the majority of human cancers are characterized by overexpression of HIF-1α (Zhong et al. 1999). However, further studies are required to exclude the possibility that a dominant-negative form of OS-9 is expressed in these tumors. Alternatively, a wide variety genetic alterations involving oncogenes and tumor suppressor genes have been shown to increase HIF-1α expression and it is possible that the optimal HIF-1α levels for growth of osteosarcomas are achieved by a compensatory downregulation of HIF-1α mediated by OS-9. Finally, properties of OS-9 unrelated to its regulation of HIF-1 may contribute to the selection of osteosarcoma cells that overexpress the protein. However, the finding that OS-9 levels are modulated in osteosarcoma cells suggests the more general principle that developmental or physiological alterations in OS-9 expression or activity may provide a means to alter the set-point of the oxygen sensing system, similar to what has been described for the PHDs (Berra et al. 2003; D'Angelo et al. 2003; Epstein et al. 2001; Hirsila et al. 2003; Metzen et al. 2003).

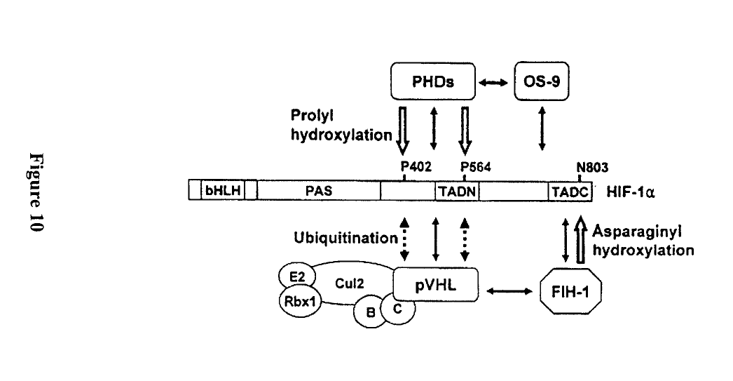

In one aspect of the present invention, the technique of yeast two-hybrid screening was used to demonstrate that FIH-1 (Mahon et al. 2001) and OS-9 are two important regulators of HIF-1. FIH-1 is the asparaginyl hydroxylase that regulates the interaction of HIF-1α with the coactivators CBP and p300, whereas OS-9 promotes the PHD-mediated prolyl hydroxylation that regulates interaction of HIF-1α with VHL. FIH-1 interacts both with HIF-1α and with VHL (Mahon et al. 2001), which also interacts with (and regulates) hydroxylated HIF-1α (FIG. 10).

Similarly, OS-9 interacts with HIF-1α and with the prolyl hydroxylases PHD 1-3, which also interact with (and regulate) HIF-1α. Thus, two ternary protein complexes have been identified with HIF-1α at their center. The formation of ternary complexes suggests cooperative binding that would insure stable protein association.

In a related aspect, such interactions may be demonstrated by Fluorescence resonance energy transfer (FRET). FRET is a distance-dependent interaction between the electronic excited states of two dye molecules in which excitation is transferred from a donor molecule to an acceptor molecule without emission of a photon. The efficiency of FRET is dependent on the inverse sixth power of the intermolecular separation (Stryer L, Haugland R P. Proc Natl Acad Sci USA (1967) 58, 719-726), making it useful over distances comparable with the dimensions of biological macromolecules. Thus, FRET is an important technique for investigating a variety of biological phenomena that produce changes in molecular proximity (e.g., see, Kawski A. Photochem Photobiol (1983) 38, 487). When FRET is used as a contrast mechanism, colocalization of proteins and other molecules can be imaged with spatial resolution beyond the limits of conventional optical microscopy (Kenworthy A K. Methods (2001) 24, 289-296).

In another related aspect, protein interaction may be demonstrated by other methods, including but not limited to, mass spectrometry, protein chip analysis, SOS recruitment systems, and RNA polymerase II based two-hybrid systems (Auerbach et al., Proteomics (2002) 2:611-623).

The HIF-1 protein and HIF-1α subunit may be any human or other mammalian protein, or fragment thereof which has the ability to bind to OS-9, PHD, FIH-1, and/or VHL protein.

A number of HIF-1α subunit proteins have been cloned. These include, but are not limited to, HIF-1α, the sequence of which is available as Genbank accession number U22431. HIF-1α subunit proteins from other species, including murine HIF-1α (accession numbers AF003695, US9496, and X95580) and rat HIF-1α (accession number Y09507).

Variants of the HIF-1 and HIF-1α subunit may be used, such as synthetic variants which have at least 45% amino acid identity to a naturally occurring HIF-1 and/or HIF-1α subunit (particularly a human HIF-1 or HIF-1α subunit), preferably at least 50%, 60%, 70%, 80%, 90%, 95%, or 98% identity.

Fragments of HIF-1 and/or HIF-1α subunit protein and its variants may be used, provided that the fragments retain the ability to interact with OS-9, PHD, FIH-1, and/or VHL. Such fragments are desirably at least 20, preferably at least 40, 50, 75, 100, 200, 250, or 400 amino acids in size. Alternately, such fragments may be 12 to 14 amino acids in size, or as small as four amino acids. Most desirably such fragments include the region 692-826 as set forth in SEQ ID NO: 2 or its equivalent regions in other HIF-1α subunit proteins. Optionally the fragments also include one or more domains of the protein responsible for transactivation. Reference herein to an HIF-1α subunit protein includes the above mentioned mutants and fragments which are functionally able to bind OS-9, PHD, FIH-1, and/or VHL protein unless the context is explicitly to the contrary.

The OS-9 protein and fragments thereof may be any human or other mammalian protein, or fragment thereof which has the ability to bind to PHD and/or HIF-1 (or HIF-1α subunit).

A number of OS-9 proteins have been cloned. These include, but are not limited to, Genbank accession numbers AB002806, JC5889, XP_{—}531650, Q13438, CAG33072, AAH06506, and AAB06495.

Variants of the OS-9 protein may be used, such as synthetic variants which have at least 45% amino acid identity to a naturally occurring OS-9 (particularly a human OS-9), preferably at least 50%, 60%, 70%, 80%, 90%, 95%, or 98% identity.

Fragments of the OS-9 protein and its variants may be used, provided that the fragments retain the ability to interact with PHD and/or HIF-1 (or HIF-1α). Such fragments are desirably at least 20, preferably at least 40, 50, 75, 100, 200, 250, or 400 amino acids in size. Alternately, such fragments may be 12 to 14 amino acids in size, or as small as four amino acids. Reference herein to a OS-9 protein includes the above mentioned mutants and fragments which are functionally able to bind PHD and/or HIF-1 (or HIF-1α) protein unless the context is explicitly to the contrary.

The PHD proteins and fragments thereof may be any human or other mammalian protein, or fragment thereof which has the ability to bind to OS-9 and/or HIF-1 (or HIF-1α subunit).

A number of PHD proteins have been cloned. These include, but are not limited to, Genbank accession numbers Q9GZT9, Q91YE3, Q91YE2, Q91UZ4, Q62630, NP_{—}848017, NP_{—}077335, Q9H6Z9, and Q96KS0.

Variants of the PHD proteins may be used, such as synthetic variants which have at least 45% amino acid identity to naturally occurring PHDs (particularly human PHDs), preferably at least 50%, 60%, 70%, 80%, 90%, 95%, or 98% identity.

Fragments of the PHD proteins and their variants may be used, provided that the fragments retain the ability to interact with OS-9 and/or HIF-1 (or HIF-1α). Such fragments are desirably at least 20, preferably at least 40, 50, 75, 100, 200, 250, or 400 amino acids in size. Alternately, such fragments may be 12 to 14 amino acids in size, or as small as four amino acids. Reference herein to a PHD protein includes the above mentioned mutants and fragments which are functionally able to bind OS-9 and/or HIF-1 (or HIF-1α) protein unless the context is explicitly to the contrary.

The VHL protein and fragments thereof may be any human or other mammalian protein, or fragment thereof which has the ability to bind to FIH-1 and/or hydroxylated HIF-1 (or hydroxylated HIF-1α subunit).

A number of VHL proteins have been cloned. These include, but are not limited to, Genbank accession numbers NP_{—}937799, NP_{—}000542, Q64259, NP_{—}033533, JC7399, AAH58831, AAP32238, AAB64200, and AAA20662.

Variants of the VHL protein may be used, such as synthetic variants which have at least 45% amino acid identity to a naturally occurring VHL (particularly a human VHL), preferably at least 50%, 60%, 70%, 80%, 90%, 95%, or 98% identity.

Fragments of the VHL protein and its variants may be used, provided that the fragments retain the ability to interact with FIH-1 and/or hydroxylated HIF-1 (or hydroxylated HIF-1α). Such fragments are desirably at least 20, preferably at least 40, 50, 75, 100, 200, 250, or 400 amino acids in size. Alternately, such fragments may be 12 to 14 amino acids in size, or as small as four amino acids. Reference herein to a VHL protein includes the above mentioned mutants and fragments which are functionally able to bind FIH-1 and/or hydroyxlated HIF-1 (or hydroxylated HIF-1α) protein unless the context is explicitly to the contrary.

The percentage homology (also referred to as identity) of DNA and amino acid sequences can be calculated using commercially available algorithms. The following programs (provided by the National Center for Biotechnology Information) may be used to determine homologies: BLAST, gapped BLAST and PSI-BLAST, which may be used with default parameters. The algorithm GAP (Genetics Computer Group, Madison, Wis.) uses the Needleman and Wunsch algorithm to align two complete sequences that maximizes the number of matches and minimizes the number of gaps. Generally, the default parameters are used, with a gap creation penalty=12 and gap extension penalty=4. Use of either of the terms “homology” and “homologous” herein does not imply any necessary evolutionary relationship between compared sequences, in keeping for example with standard use of terms such as “homologous recombination” which merely requires that two nucleotide sequences are sufficiently similar to recombine under the appropriate conditions.

In one embodiment, the invention relates to a method of identifying a compound which modulates the hypoxia-inducible pathway mediated induction of protein and/or gene expression. In one aspect, a cell line stably or transiently transfected with a vector comprising a hypoxia/HIF-inducible promoter operably linked to a reporter gene is used to detect compounds which modulate the expression of genes or any proteins modulated by hypoxia and/or HIF interaction with OS-9. In one aspect of this method, cell lines comprising a hypoxia/HIF-inducible promoter operably linked to a reporter gene can be used to detect compounds which modulate the expression of the reporter gene as an indirect measure of the modulation of OS-9/HIF-1 interaction. In one embodiment, the expression of the reporter gene can be readily detected, e.g., by a simple calorimetric assay. Other genes which can be detected by other techniques such as enzymatic or fluorometric assays can be used as the reporter gene.

Compounds that test positive in the modulator identification assays of the invention are those that modulate the expression of the reporter gene. For example, cells are incubated with a test compound under specified conditions and compared to cells incubated under identical conditions except for the absence of that compound. A comparison between reporter gene expression with the test compound and reporter gene expression from the no-compound assay allows one to determine if the test compound is positive. Those test compounds which alter expression levels of the reporter gene compared to the no-compound (or other appropriate control) have tested “positive.”

Materials that test positive in the assays of the invention are useful for modulating the OS-9/HIF pathway which is associated with a variety of clinical significant conditions, i.e., cancer, ischemia, and the like.

In certain aspects of this embodiment, the cells are lysed or further processed before OS-9 is contacted with the test compound. In any case, after the test compound is incubated for a selected period of time with the lysate, the reaction mixture is assayed for level of reporter gene expression. In particularly useful aspects of this embodiment, the reporter gene expresses a protein that is readably detectable, e.g., an enzyme which catalyzes a reaction that is detected by a simple calorimetric assay or by other means such as monoclonal antibody detection. Examples of reporter genes useful in the invention include, but are not limited to, luciferase, β-galactosidase, alkaline phosphatase, green fluorescent protein, etc.

Small molecule entities or test compounds which may be useful in the present invention include compounds which may specifically interact with OS-9. Examples of such molecules include, but are not limited to, drugs or therapeutic compounds; toxins, such as those present in the venoms of poisonous organisms, including certain species of spiders, snakes, scorpions, dinoflagellates, marine snails and bacteria; growth factors, such as NGF, PDGF, TGF and TNF; cytokines; and bioactive peptides.

The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, Anticancer Drug Design (1997) 12:145.

Libraries of chemical and/or biological mixtures, such as fungal, bacterial, or algal extracts, are known in the art and can be screened with any of the assays of the invention. Examples of methods for the synthesis of molecular libraries can be found in the art, for example in: DeWitt et al., Proc Natl Acad Sci USA (1993) 90:6909; Erb et al., Proc Natl Acad Sci USA (1994) 91:11422; Zuckermann et al., J Med Chem (1994) 37:2678; Cho et al., Science (1993) 261:1303; Carrell et al., Angew Chem Int Ed Engl (1994) 33:2059; Carrell et al., Angew Chem Int Ed Engl (1994) 33:2061; and Gallop et al., J Med Chem (1994) 37:1233.

Libraries of compounds may be presented in solution (e.g., Houghten, Biotechniques (1992) 13:412-421), or on beads (Lam, Nature (1991) 354:82-84), on chips (Fodor, Nature (1993) 364:555-556), bacteria (Ladner, U.S. Pat. No. 5,223,409), spores (Ladner, U.S. Pat. No. 5,233,409), plasmids (Cull et al., Proc Natl Acad Sci USA (1992) 89:1865-1869) or on phage (Scott and Smith, Science (1990) 249:386-390; Devlin, Science (1990) 249:404-406; Cwirla et al., Proc Natl Acad Sci USA (1990) 87:6378-6382; Felici, J Mol Biol (1991) 222:301-310; Ladner, U.S. Pat. No. 5,233,409.).

A variety of host-expression vector systems can be used to express the nucleotide sequences of the invention. Where the peptide or polypeptide can exist, or has been engineered to exist, as a soluble or secreted molecule, the soluble peptide or polypeptide can be recovered from the culture media. Such expression systems also encompass engineered host cells that express proteins, or functional equivalents, in situ. Purification or enrichment of a protein of the instant invention from such expression systems can be accomplished using appropriate detergents and lipid micelles and methods well known to those skilled in the art. However, such engineered host cells themselves may be used in situations where it is important not only to retain the structural and functional characteristics of the encoded protein, but to assess biological activity, e.g., in drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors; yeast (e.g., Saccharomyces, Pichia) transformed with recombinant yeast expression vectors; insect cell systems infected with recombinant virus expression vectors (e.g., baculovirus); or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5 K promoter).

In bacterial systems, a number of expression vectors may be advantageously selected depending upon the use intended for the product being expressed. For example, when a large quantity of a protein is to be produced for the generation of pharmaceutical compositions, or for raising antibodies, vectors that direct the expression of high levels of fusion protein products that are readily purified may be desirable. Such vectors include, but are not limited, to the E. coli expression vector pUR278 (Ruther et al., EMBO J (1983) 2:1791), in which a coding sequence may be ligated individually into the vector in frame with the lacZ coding region so that a fusion protein is produced; pIN vectors (Inouye and Inouye, Nucleic Acids Res (1985) 13:3101-3109; Van Heeke & Schuster, J Biol Chem (1989) 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target expression product can be released from the GST moiety.

Alternatively, any fusion protein can be readily purified by utilizing an antibody specific for the fusion protein being expressed. For example, a system described by Janknecht et al. allows for the ready purification of non-denatured fusion proteins expressed in human cell lines (Janknecht et al., Proc Natl Acad Sci USA (1991) 88:8972-8976). In this system, the sequence of interest is subcloned into a vaccinia recombination plasmid such that the open reading frame of the sequence is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The invention includes entities which may have been modified or conjugated to include, for example, a light-generating fusion protein of the invention. Such conjugated or modified entities are referred to as light-emitting entities, or simply conjugates. The conjugates themselves may take the form of, for example, molecules, macromolecules, particles, microorganisms, or cells. The methods used to conjugate a light-generating fusion protein to an entity depend on the nature of the light-generating fusion protein and the entity.

In an insect system, Autographa californica nuclear polyhedrosis virus (AcNPV) is used as a vector to express foreign polynucleotide sequences. The virus grows in Spodoptera frugiperda cells. A coding sequence can be cloned individually into non-essential regions (for example the polyhedrin gene) of the virus and placed under control of an AcNPV promoter (for example the polyhedrin promoter). Successful insertion of a coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted sequence is expressed (e.g., see Smith et al., J Virol (1983) 46:584; Smith, U.S. Pat. No. 4,215,051).

In mammalian host cells, a number of viral-based expression systems may be utilized. In cases where an adenovirus is used as an expression vector, the nucleotide sequence of interest may be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing a product in infected hosts (e.g., See Logan & Shenk, Proc Natl Acad Sci USA (1984) 81:3655-3659). Specific initiation signals may also be required for efficient translation of inserted nucleotide sequences. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of a coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bitter et al., Methods in Enzymol (1987) 153:516-544).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, 3T3, W138, and in particular, human cell lines.

For long-term, high-yield production of recombinant proteins, stable expression is preferred. For example, cell lines that stably express exogenous sequences can be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the product. Such engineered cell lines may be particularly useful in screening and evaluation of compounds that affect the endogenous activity of the product.

A number of selection systems may be used, including but not limited to the herpes simplex virus thymidine kinase (Wigler et al., Cell (1977) 11:223), hypoxanthine-guanine phosphoribosyltransferase (Szybalska & Szybalski, Proc Natl Acad Sci USA (1962) 48:2026), and adenine phosphoribosyltransferase (Lowy et al., Cell (1980) 22:817) genes, which can be employed in tk-, hgprt-, or aprt- cells, respectively. Also, antimetabolite resistance can be used as the basis of selection for the following genes: dhfr, which confers resistance to methotrexate (Wigler et al., Natl Acad Sci USA (1980) 77:3567; O'Hare et al., Proc Natl Acad Sci USA (1981) 78:1527); gpt, which confers resistance to mycophenolic acid (Mulligan & Berg, Proc Natl Acad Sci USA (1981) 78:2072); neo, which confers resistance to the aminoglycoside G-418 (Colberre-Garapin et al., J Mol Biol (1981) 150:1); and hygro, which confers resistance to hygromycin (Santerre et al., Gene (1984) 30:147).

In one embodiment, OS-9 activity is modulated by antisense nucleic acids. The present invention provides for the therapeutic or prophylactic use of nucleic acids comprising at least six nucleotides that are antisense to the genes or cDNAs encoding OS-9 or portions thereof. As used herein, OS-9 “antisense” nucleic acids refer to nucleic acids capable of hybridizing by virtue of some sequence complementarity to a portion of an RNA (preferably mRNA) encoding OS-9. The antisense nucleic acids may be complementary to a coding and/or noncoding region of an mRNA encoding OS-9. Such antisense nucleic acids have utility as compounds that prevent OS-9 expression, and can be used in the treatment for example, of ischemic conditions. The antisense nucleic acids of the invention are double-stranded or single-stranded oligonucleotides, RNA, or DNA, or a modification or derivative thereof, and can be directly administered to a cell or produced intracellularly by transcription of exogenous, introduced sequences.

The invention further provides pharmaceutical compositions comprising a therapeutically effective amount of OS-9 antisense nucleic acids, and a pharmaceutically acceptable carrier, vehicle, or diluent.

The OS-9 antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. In addition, the antisense molecules may be polymers that are nucleic acid mimics, such as PNA, morpholino oligos, and LNA. Other types of antisence molecules include short double-stranded RNAs, known as siRNAs, and short hairpin RNAs, and long dsRNA (>50 bp but usually <500 bp).

In another embodiment, OS-9 expression is inhibited by a short interfering RNA (siRNA) through RNA interference (RNAi) or post-transcriptional gene silencing (PTGS) (see, for example, Ketting et al., Genes Develop (2001) 15:2654-2659). siRNA molecules can target homologous mRNA molecules for destruction by cleaving the mRNA molecule within the region spanned by the siRNA molecule. Accordingly, siRNAs capable of targeting and cleaving homologous OS-9 mRNA are useful for treating, for example, ischemic disorders.

In another embodiment, ischemic disorders may be treated in a subject suffering from such disease by decreasing the level of OS-9 activity by using ribozyme molecules designed to catalytically cleave gene mRNA transcripts encoding OS-9, preventing translation of target gene mRNA and, therefore, expression of the gene product.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246. Ribozymes that cleave mRNA at site-specific recognition sequences can be used to destroy mRNA encoding OS-9. In a related aspect, hammerhead ribozymes can be used. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art. The ribozymes of the present invention also include RNA endoribonucleases (hereinafter “Cech-type ribozymes”) such as the one that occurs naturally in Tetrahymena thermophila (known as the IVS, or L-19 IVS RNA). The Cech-type ribozymes have an eight base pair active site that hybridizes to a target RNA sequence where after cleavage of the target RNA takes place.

In one embodiment, OS-9 activity is modulated by using antibodies. For the production of antibodies, various host animals may be immunized by injection with OS-9, an OS-9 peptide, truncated OS-9 polypeptides, functional equivalents of OS-9 or mutated variant of OS-9. Such host animals may include but are not limited to pigs, rabbits, mice, goats, and rats, to name but a few. Various adjuvants may be used to increase the immunological response, depending on the host species, including but not limited to Freund's adjuvant (complete and incomplete), mineral salts such as aluminum hydroxide or aluminum phosphate, chitosan, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and or coupling with molecules such as keyhole limpet hemocyanin, tetanus toxoid, diphtheria toxoid, ovalbumin, cholera toxin or fragments thereof. Polyclonal antibodies are heterogeneous populations of antibody molecules derived from the sera of the immunized animals.

Monoclonal antibodies, which are homogeneous populations of antibodies to a particular antigen, can be obtained by any technique which provides for the production of antibody molecules by continuous cell lines in culture. These include, but are not limited to, the hybridoma technique of Kohler and Milstein, (Nature (1975) 256:495-497; and U.S. Pat. No. 4,376,110), the human B-cell hybridoma technique (Kosbor et al., Immunology Today (1983) 4:72; Cole et al., Proc Natl Acad Sci USA (1983) 80:2026-2030), and the EBV-hybridoma technique (Cole et al., Monoclonal Antibodies And Cancer Therapy, (1985) Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., Proc Natl Acad Sci USA (1984) 81:6851-6855; Neuberger et al., Nature (1984) 312:604-608; Takeda et al., Nature (1985) 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region (see U.S. Pat. Nos. 6,075,181, 5,877,397 and 6,150,584, which are herein incorporated by reference in their entirety).

Alternatively, techniques described for the production of single chain antibodies (U.S. Pat. No. 4,946,778; Bird, Science (1988) 242:423-426; Huston et al., Proc Natl Acad Sci. USA (1988) 85:5879-5883; and Ward et al., Nature (1989) 341:544-546) can be adapted to produce single chain antibodies against OS-9 expression products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include, but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries maybe constructed (Huse et al., Science (1989) 246:1275-1281) to allow rapid and easy identification of monoclonal Fab fragments with the desired specificity.

The precise format of the screening assays may be varied using routine skill and knowledge. Where assays of the invention are performed within cells, the cells may be treated to provide or enhance a normoxic environment. By “normoxic” it is meant levels of oxygen similar to those found in normal air, e.g. about 21% O₂ and 5% CO₂, the balance being nitrogen. Of course, these exact proportions do not have to be used, and may be varied independently of each other. Generally a range of from 10-30% oxygen, 1-10% CO₂ and a balance of nitrogen or other relatively inert and non-toxic gas may be used. Normoxia may be induced or enhanced in cells, for example by culturing the cells in the presence of hydrogen peroxide.

Alternatively or by way of controls, cells may also be cultured under hypoxic conditions. By “hypoxic” it is meant an environment with reduced levels of oxygen. Most preferably oxygen levels in cell culture will be 0.1 to 1.0% for the provision of a hypoxic state. Hypoxia may be induced in cells simply by culturing the cells in the presence of lowered oxygen levels. The cells may also be treated with compounds which mimic hypoxia and cause up regulation of HIF-1α subunit expression. Such compounds include iron chelators, cobalt (II), nickel (II) or manganese (II), all of which may be used at a concentration of 20 to 500 μM. such as 100 μM. Iron chelators include desferrioxamine, O-phenanthroline or hydroxypyridinones (e.g., 1,2-diethyl hydroxypyridinone (CP94) or 1,2-dimethyl hydroxypyridinone (CP20)).

Cells in which assays of the invention may be preformed include eukaryotic cells, such as yeast, insect, mammalian primate and human cells. Mammalian cells may be primary cells or transformed cells, including tumor cell lines. The cells may be modified to express or not to express other proteins which are known to interact with HIF-1 (α subunit proteins and VHL protein, for example Flongin C and Elongin B proteins in the case of VHL and ARNT protein, in the case of HIF-1α subunit protein).

In cell free systems such additional proteins may be included, for example by being provided by expression from suitable recombinant expression vectors.

The amount of putative modulator compound which may be added to an assay of the invention will normally be determined by trial and error depending upon the type of compound used. Modulator compounds may be those which either agonize or antagonize the interaction.

Modulator compounds which may be used may be natural or synthetic chemical compounds used in drug screening programs. Extracts of plants which contain several characterized or uncharacterized components may also be used.

According to one aspect of the invention, a method for treating a subject having a condition characterized by an abnormal mammalian cell proliferation is provided. As used herein, subject means a mammal including humans, nonhuman primates, dogs, cats, sheep, goats, horses, cows, pigs and rodents. An abnormal mammalian cell proliferation disorder or condition, as used herein, refers to a localized region of cells (e.g., a tumor) which exhibit an abnormal (e.g., increased) rate of division as compared to their normal tissue counterparts.

Conditions characterized by an abnormal mammalian cell proliferation, as used herein, include, but are not limited to, conditions involving solid tumor masses of benign, pre-malignant or malignant character. Although not wishing to be bound by a particular theory or mechanism, some of these solid tumor masses arise from at least one genetic mutation, some may display an increased rate of cellular proliferation as compared to the normal tissue counterpart, and still others may display factor independent cellular proliferation. Factor independent cellular proliferation is an example of a manifestation of loss of growth control signals which some, if not all, tumors or cancers undergo.

According to another aspect of the invention, methods are provided for inhibiting angiogenesis in disorders having a pathology which requires angiogenesis. Angiogenesis is defined as the formation of new blood vessels. One subset of these disorders is conditions characterized by abnormal mammalian cell proliferation. Another subset is non-cancer conditions including diabetic retinopathy, neovascular glaucoma and psoriasis.

In some embodiments, the methods of the invention are aimed at inhibiting tumor angiogenesis. Tumor angiogenesis refers to the formation of new blood vessels in the vicinity or within a tumor mass. Solid tumor cancers require angiogenesis particularly for oxygen and nutrient supply. It has been previously shown that inhibition of angiogenesis in solid tumor can cause tumor regression in animal models. Thus in one aspect, the invention relates to a method for inhibiting angiogenesis by inhibiting the proliferation, migration or activation of endothelial cells and fibroblasts, provided this angiogenesis is unrelated to wound healing in response to injury, infection or inflammation.

Thus in certain embodiments, the methods of the invention are intended for the treatment of diseases and processes that are mediated by angiogenesis including, but not limited to, hemangioma, solid tumors, tumor metastasis, benign tumors, for example hemangiomas, acoustic neuromas, neurofibromas and trachomas, Osler-Webber Syndrome, telangiectasia, myocardial angiogenesis, angiofibroma, plaque neovascularization, coronary collaterals, ischemic limb angiogenesis, comneal diseases, rubiosis, neovascular glaucoma, diabetic retinopathy, retrolental fibroplasia, diabetic neovascularization, macular degeneration, keloids, ovulation, menstruation, and placentation.

The circulatory system serves an important role in the transport of nutrients, proteins, hormones, and other vital molecules that are necessary to maintain life. Blood vessels, which form an intricate network of pathways, represent an integral component of the circulatory system. In mammalian species, the internal surface of a blood vessel lumen is comprised of endothelial cells. These endothelial cells impart a smooth and low resistance quality to the lumenal surface. Critical to the free flow and transport of blood and blood constituents, the smooth and nonadhesive internal surface of the blood vessel increases the ease with which fluid flows. Without a smooth internal surface, blood vessels would become obstructed due to the formation of thrombi or other blockages at “sticky” locations on the internal walls. Complete or even partial blood vessel blockage would cause restriction of blood flow, thereby compromising the viability of living tissue served by the vessel. Thus, endothelial cells represent an important structural component of blood vessels and also provide blood vessels with a smooth internal surface.

The formation of blood vessels in vivo takes place in response to stimuli, which are provided in the form of specialized growth factors. These growth factors induce mitosis in cells already present in blood vessels. The new cells may replace nearby damaged cells, or the new cells may arrange themselves such that new blood vessels are formed. The process of growing blood vessels from endothelial cells is termed “angiogenesis,” which results in, among other characteristics, the vascularization of tissue.

Angiogenesis has become a central theme in promoting our understanding of how tissue grows. As indicated above, endothelial cell proliferation is not only desirable, but also necessary to carry out a number of physiological processes, for example the in utero formation of tissues and organs. Conditions that can be treated in accordance with this method of the invention (administration by any route, preferably oral administration) are conditions characterized by insufficient vascularization (or predisposition thereto) of the affected tissue, i.e., conditions in which neovascularization (rather than increases in nitric oxide (NO)-mediated vasodilation) is needed to achieve sufficient vascularization in the affected tissue, and that are selected from the following group of conditions: diabetic ulcers, gangrene, surgical or other wounds requiring ncovascularization to facilitate healing; Buerger's syndrome; hypertension; ischemic diseases including, for example, cerebrovascular ischemia, renal ischemia, pulmonary ischemia, limb ischemia, ischemic cardiomyopathy, myocardial ischemia, ischemia of tissues such as, for example, muscle, brain, kidney and lung; and other conditions characterized by a reduction in microvasculature. The preferred method of treatment further includes the step of detecting angiogenesis in the affected tissue following treatment. Exemplary tissues in which angiogenesis can be promoted in accordance with this method of the invention include: hypertension; ulcers (e.g., diabetic ulcers); surgical wounds; ischemic tissue, i.e., a tissue having a deficiency in blood as the result of an ischemic disease including, for example, muscle, brain, kidney and lung; ischemic diseases including, for example, cerebrovascular ischemia, renal ischemia, pulmonary ischemia, limb ischemia, ischemic cardiomyopathy and myocardial ischemia.

The pharmaceutical compositions of the invention comprise the novel agents combined with a pharmaceutically acceptable carrier. The term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Although any route of administration may be used, parenteral administration, i.e., administration by injection, is preferred. Injectable formulations can be prepared in conventional forms, either as liquid solutions or suspensions; as solid forms suitable for solubilization or suspension in liquid prior to injection; or as emulsions. Preferably, sterile injectable suspensions are formulated according to techniques known in the art using suitable pharmaceutically acceptable carriers and other optional components as discussed above.

Parenteral administration may be carried out in any number of ways, but it is preferred that the use of a syringe, catheter, or similar device, be used to effect parenteral administration of the formulations described herein. The formulation may be injected systemically such that the active agent travels substantially throughout the entire bloodstream. Also, the formulation may also be injected locally to a target site, i.e., injected to a specific portion of the body for which inhibition of angiogenesis is desired. An advantage of local administration via injection is that it limits or avoids exposure of the entire body to the active agent. It must be noted that in the present context, the term local administration includes regional administration, e.g., administration of a formulation directed to a portion of the body through delivery to a blood vessel serving that portion. Local delivery may be direct, i.e., intratumoral. Local delivery may also be nearly direct, i.e., intralesional or intraperitoneal, that is, to an area that is sufficiently close to a tumor so that the active agent exhibits the desired pharmacological activity. Thus, when local delivery is desired, the pharmaceutical formulations are preferably delivered intralesionally, intratumorally, or intraperitoneally.

It is intended that, by local delivery of the presently described pharmaceutical formulations, a higher concentration of the active agent may be retained at the target site. There are several advantages to having high concentrations delivered directly at the target site. First, since the active agent is localized, there is less potential for toxicity to the subject since minimal systemic exposure occurs. Second, drug efficacy is improved since the target site is exposed to higher concentrations of drug. Third, relatively fast delivery ensures both solubility of the drug and little or no degradation of the active agent before reaching the target site. Fourth, the method is relatively noninvasive, which is ideal for unresectable tumors such as brain tumors, liver tumors, and pancreatic tumors.

With local administration, it is preferred that the pharmaceutical formulations of the present invention be directed to the target area with the assistance of computerized tomography (CT), ultrasound, or similar method in order to ensure correct placement. Once the initial dose is administered, the subject may be given other doses either immediately or after a period of time. Such a dosing schedule is easily determined by one of ordinary skill in the art once the nature of the condition, disorder, or disease, strength of the subject, expected effects of the formulation, and so forth, are taken into consideration.

The present invention also encompasses gene therapy whereby a polynucleotide encoding angiogenic modulating nucleic acids, proteins or a mutant, fragment, or fusion protein thereof, is introduced and regulated in a subject. Various methods of transferring or delivering DNA to cells for expression of the gene product, otherwise referred to as gene therapy, are disclosed in Gene Transfer into Mammalian Somatic Cells in vivo, N. Yang, Crit Rev Biotechn (1992) 12(4):335-356, which is hereby incorporated by reference. Gene therapy encompasses incorporation of DNA sequences into somatic cells or germ line cells for use in either ex vivo or in vivo therapy. Gene therapy functions to replace genes, augment normal or abnormal gene function, and to combat infectious diseases and other pathologies.

Strategies for treating these medical problems with gene therapy include therapeutic strategies such as identifying the defective gene and then adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene such as that encoding an antisense OS-9 RNA may be placed in a subject to serve as a medicament for a cardiovascular disorder by promoting angiogenesis.

Many protocols for transfer of nucleic acids are envisioned in the present invention. Transfection of promoter sequences are envisioned as a method of gene therapy. An example of this technology is found in Transkaryotic Therapies, Inc., of Cambridge, Mass., using homologous recombination to insert a “genetic switch” that turns on an erythropoietin gene in cells. See Genetic Engineering News, Apr. 15, 1994. Such “genetic switches” could be used to activate the angiogenic/anti-angiogenic gene products in cells not normally expressing those products.

Gene transfer methods for gene therapy fall into three broad categories: physical (e.g., electroporation, direct gene transfer, and particle bombardment), chemical (e.g., lipid-based carriers, or other non-viral vectors) and biological (e.g., virus-derived vector and receptor uptake). For example, non-viral vectors may be used which include liposomes coated with DNA. Such liposome/DNA complexes may be directly injected intravenously into the subject. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic nucleic acid.

Gene therapy methodologies can also be described by delivery site. Fundamental ways to deliver genes include ex vivo gene transfer, in vivo gene transfer, and in vitro gene transfer. In ex vivo gene transfer, cells are taken from the subject and grown in cell culture. The DNA is transfected into the cells, the transfected cells are expanded in number and then reimplanted in the subject. In in vitro gene transfer, the transformed cells are cells growing in culture, such as tissue culture cells, and not particular cells from a particular subject. These “laboratory cells” are transfected, the transfected cells are selected and expanded for either implantation into a subject or for other uses.

In vivo gene transfer involves introducing the DNA into the cells of the subject when the cells are within the subject. Methods include using virally mediated gene transfer using a noninfectious virus to deliver the gene in the subject or injecting naked DNA into a site in the subject and the DNA is taken up by a percentage of cells in which the gene product is expressed. Additionally, the other methods described herein, such as use of a “gene gun,” may be used for in vitro insertion of the nucleic acid controlling production of the desired gene product.

Chemical methods of gene therapy may involve a lipid based compound, not necessarily a liposome, to transfer the DNA across the cell membrane. Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the subject to provide the gene product from the inserted DNA.

Viral vectors have also been used to insert genes into cells using in vivo protocols. To direct the tissue-specific expression of foreign genes, cis-acting regulatory elements or promoters that are known to be tissue-specific can be used. Alternatively, this can be achieved using in situ delivery of DNA or viral vectors to specific anatomical sites in vivo. For example, gene transfer to blood vessels in vivo was achieved by implanting in vitro transduced endothelial cells in chosen sites on arterial walls. The virus infected surrounding cells which also expressed the gene product. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors that have been used for gene therapy protocols include, but are not limited to, retroviruses, other RNA viruses such as poliovirus or Sindbis virus, adenovirus, adeno-associated virus, herpes viruses, SV40, vaccinia, and other DNA viruses. Replication-defective murine retroviral vectors are the most widely utilized gene transfer vectors. Murine leukemia retroviruses are composed of a single strand RNA complexed with a nuclear core protein and polymerase (pol) enzymes, encased by a protein core (gag) and surrounded by a glycoprotein envelope (env) that determines host range. The genomic structure of retroviruses include the gag, pol, and env genes enclosed at by the 5′ and 3′ long terminal repeats (LTR). Retroviral vector systems exploit the fact that a minimal vector containing the 5′ and 3′ LTRs and the packaging signal are sufficient to allow vector packaging, infection, and integration into target cells providing that the viral structural proteins are supplied in trans in the packaging cell line. Fundamental advantages of retroviral vectors for gene transfer include efficient infection and gene expression in most cell types, precise single copy vector integration into target cell chromosomal DNA, and ease of manipulation of the retroviral genome.

The adenovirus is composed of linear, double stranded DNA complexed with core proteins and surrounded with capsid proteins. Advances in molecular virology have led to the ability to exploit the biology of these vehicles to create vectors capable of transducing novel genetic sequences into target cells in vivo. Adenoviral-based vectors will express gene product proteins at high levels. Adenoviral vectors have high efficiencies of infectivity, even with low titers of virus. Additionally, the virus is fully infective as a cell free virion so injection of producer cell lines is not necessary. Another potential advantage to adenoviral vectors is the ability to achieve long term expression of heterologous genes in vivo.

Mechanical methods of DNA delivery include fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” and inorganic chemical approaches such as calcium phosphate transfection. Particle-mediated gene transfer methods were first used in transforming plant tissue. With a particle bombardment device, or “gene gun,” a motive force is generated to accelerate DNA-coated high density particles (such as gold or tungsten) to a high velocity that allows penetration of the target organs, tissues or cells. Particle bombardment can be used in in vitro systems, or with ex vivo or in vivo techniques to introduce DNA into cells, tissues or organs. Another method, ligand-mediated gene therapy, involves complexing the DNA with specific ligands to form ligand-DNA conjugates, to direct the DNA to a specific cell or tissue.

It has been found that injecting plasmid DNA into muscle cells yields high percentage of the cells which are transfected and have sustained expression of marker genes. The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Electroporation for gene transfer uses an electrical current to make cells or tissues susceptible to electroporation-mediated mediated gene transfer. A brief electric impulse with a given field strength is used to increase the permeability of a membrane in such a way that DNA molecules can penetrate into the cells. This technique can be used in in vitro systems, or with ex vivo or in vivo techniques to introduce DNA into cells, tissues or organs.

Carrier mediated gene transfer in vivo can be used to transfect foreign DNA into cells. The carrier-DNA complex can be conveniently introduced into body fluids or the bloodstream and then site-specifically directed to the target organ or tissue in the body. Both liposomes and polycations, such as polylysine, lipofectins or cytofectins, can be used. Liposomes can be developed which are cell specific or organ specific and thus the foreign DNA carried by the liposome will be taken up by target cells. Injection of immunoliposomes that are targeted to a specific receptor on certain cells can be used as a convenient method of inserting the DNA into the cells bearing the receptor. Another carrier system that has been used is the asialoglycoportein/polylysine conjugate system for carrying DNA to hepatocytes for in vivo gene transfer.

The transfected DNA may also be complexed with other kinds of carriers so that the DNA is carried to the recipient cell and then resides in the cytoplasm or in the nucleoplasm. DNA can be coupled to carrier nuclear proteins in specifically engineered vesicle complexes and carried directly into the nucleus.

Gene regulation of the exogenous nucleic acids may be accomplished by administering compounds that bind to the gene encoding one of the protein of interest, or control regions associated with the gene, or its corresponding RNA transcript to modify the rate of transcription or translation. Additionally, cells transfected with a DNA sequence of interest may be administered to a subject to provide an in vivo source of gene products encoded by the DNA. For example, cells may be transfected with a vector containing a nucleic acid sequence encoding the angiogenesis promoting gene product. The transfected cells may be cells derived from the subject's normal tissue, the subject's diseased tissue, or may be non-subject cells.

For example, tumor cells removed from a subject can be transfected with a vector capable of expressing the gene product of the present invention, and re-introduced into the subject. The transfected tumor cells produce levels of the gene product in the subject that inhibit the growth of the tumor. Subjects may be human or non-human animals. Cells may also be transfected by non-vector, or physical or chemical methods known in the art such as electroporation, ionoporation, or via a “gene gun.” Additionally, the DNA may be directly injected, without the aid of a carrier, into a subject. In particular, the DNA may be injected into skin, muscle or blood.

The gene therapy protocol for transfecting the nucleic acids into a subject may either be through integration of the gene product DNA into the genome of the cells, into minichromosomes or as a separate replicating or non-replicating DNA construct in the cytoplasm or nucleoplasm of the cell. Expression of the gene product may continue for a long-period of time or may be reinjected periodically to maintain a desired level of the gene product(s) in the cell, the tissue or organ or a determined blood level.

The amount of the active agent administered will, of course, be dependent on the subject being treated, the subject's weight, the manner of administration, and the judgment of the prescribing physician. The amount of the active agent administered, for example, will be an effective angiogenesis-inhibiting/angiogenesis-promoting amount. Preferably, the active agent is administered in an amount of from about 0.0001 mg/kg to about 200 mg/kg (milligrams of drug per kilogram body weight of the subject), more preferably from about 0.0001 mg/kg to 120 mg/kg, still more preferably from about 0.0001 mg/kg to about 15 mg/kg, yet still more preferably from about 0.5 mg/kg to about 15 mg/kg, and most preferably from about 1 mg/kg to about 13 mg/kg. Depending on the subject's response, additional dosages within this range may be administered.

The total amount of the formulation delivered to the subject will depend upon, inter alia, the condition, disease, or disorder being treating, the type of the subject, e.g., human or animal, and the subject's body weight. Generally, however, total volumes of between about 0.1 ml to about 60 ml, and more preferably between about 0.5 ml to about 30 ml, of formulation are used. Most preferably, the total volume administered of the presently described pharmaceutical formulation is from about 1.0 ml to about 15 ml.

The following examples are intended to illustrate but not limit the invention.

### EXAMPLES

Materials and Methods

Tissue Culture

Human 293 and Hep3B cells were maintained in Dulbecco's modified Eagle's medium and modified Eagle's medium with Earle's salts, respectively, supplemented with 10% fetal bovine serum, 100 units/ml penicillin, and 100 μg/ml streptomycin (Invitrogen). Cells were maintained at 37° C. in a humidified 5% CO₂, 95% air incubator. For hypoxic exposures, cells were placed in a modulator incubator chamber (Billups-Rothenberg) that was flushed with a gas mixture consisting of 1% O₂, 5% CO₂, with balance N₂, sealed, and incubated at 37° C.
Yeast Two-hybrid System Vectors and Library Screening

Bait vector pGAL4-HIF-1α (576-826) was constructed by PCR amplification of HIF-1α cDNA sequences (using forward and reverse primers containing NdeI and BamHI restriction sites, respectively), restriction endonuclease digestion, and ligation into the vector pAS2-1 (Mahon et al. 2001). Prey vectors were derived from a human brain MATCHMAKER cDNA library cloned into plasmid pACT II (Clontech). Interaction of bait and prey proteins within yeast cells reconstitutes active GAL4, resulting in transcription of genes that mediate histidine auxotrophy (his⁺) and α-gal activity. To screen for such cells, Saccharomyces cerevisiae strain Y190 was transformed by the LiAc/PEG method. YPD medium was inoculated with overnight culture and grown to OD₆₀₀=0.5. Cells were pelleted, resuspended in 8 ml of TE/LiAc solution, and exposed to 300 mg of pGAL4-HIF-1α (576-826), 600 mg of pACT II/human brain cDNA, and 20 mg of herring testes DNA (Clontech). The cells were agitated at 30° C. for 30 min, mixed with 7 ml of DMSO, heat-shocked for 15 min at 30° C., and plated onto media lacking tryptophan, leucine, and histidine and supplemented with 15 mM 3-amino-1,2,4-triazole and X-α-gal.

Purification of his⁺/α-gal-expressing clones and identification offalse positives

his⁺ and α-gal-expressing colonies were subjected to three rounds of colony purification. An individual colony was selected from the final master plate and grown in liquid medium lacking leucine to select for the presence of the prey vector. The culture was spread onto medium lacking leucine and supplemented with 10 μg/ml cycloheximide to cure clones of the bait vector and identify prey vectors encoding a protein capable of autonomous activation of the α-gal reporter gene (i.e., false positives). Individual colonies were picked from cycloheximide plates and grown in liquid culture lacking leucine. The prey vector was isolated by the glass bead method (Hoffman and Winston 1987) for transformation of E. coli DH5α cells and plasmid DNA isolation. Retransformation of yeast strain Y190 with the bait and prey vectors was performed to demonstrate that the resulting transformants were again his⁺ and expressed α-gal.
Construction of OS-9 Expression Vector

The open reading frame of OS-9 cDNA was amplified from an EST clone (AB002806) using a forward primer that encoded the hemagglutinin epitope (HA) and Kozak consensus sequence for translation initiation. The PCR product was ligated into pCR3.1 (Invitrogen).
In Vitro Interaction (GST Pull-down) Assays

To prepare GST fusion proteins, E. coli BL21-Gold(DE3)pLysS (Stratagene) was transformed with a pGEX expression vector and treated for 4 h with 0.5 mM isopropyl-D-thiogalactoside. Pelleted cells were lysed by sonication in PBS containing 1% Triton X-100 and Complete protease inhibitor cocktail (Roche). After centrifugation, supernatants were applied to glutathione-Sepharose 4B beads (Amersham Pharmacia Biotech). GST fusion proteins were eluted with 10 mM reduced glutathione in 50 mM Tris-HCl (pH 8.0) and stored at −80° C. The concentration and purity of eluates were determined by the Bradford method and by SDS-PAGE. [³⁵S]methionine-labeled proteins were generated in reticulocyte lysates with plasmids encoding HA-OS-9 or FLAG-VHL using the TNT T7 coupled transcription/translation system (Promega). Ten μl of in vitro-translated ³⁵S-labeled protein was mixed with 4 μg of GST or GST-HIF-1α fusion protein (Mahon et al. 2001) in a final volume of 200 μl of PBS-T binding buffer (Dulbecco's PBS [pH 7.4], 0.1% Tween-20). The binding reaction was performed at 4° C. for 2 h with rotation followed by the addition of 20 ml pre-washed glutathione-Sepharose 4B beads. After 30 min of mixing on a rotator, the beads were washed three times with PBS-T. Proteins were eluted in Laemmli sample buffer and analyzed by SDS-PAGE followed by autoradiography.
Transfection Assays

293 or Hep3B cells were seeded onto 24-well plates at 8×10⁴ cells per well of a 24-well plate. The following day, the cells were transfected with plasmid DNAs using Fugene-6 (Roche) for 293 and Lipofectamine Plus (Invitrogen) for Hep3B cells. After 24 h, the cells were exposed to 20% or 1% O₂ for 24 h. Cells were lysed, and the luciferase activities were determined by multi-well luminescence reader (PerkinElmer), using the Dual-Luciferase Reporter Assay System (Promega). For p2.1 reporter assay, cells were co-transfected with 15 ng of control reporter pSV-Renilla, 135 ng of HIF-1 reporter p2.1, and expression vector encoding HA-OS-9, HIF-1α, or empty vector. Unless indicated otherwise, 200 ng of HIF-1α, 400 ng of HA-OS-9, 1 ng of PHD2, or 1 ng of HIF-1α triple mutant P402A/P564A/N803A (HIF-1α TM) expression vector was used for co-transfection. For pGalA reporter assays, Hep3B cells were co-transfected with 12.5 ng of pSV-Renilla, reporter 200 ng of pG5E1bLuc, 100 ng of expression vector encoding the GAL4 DNA-binding domain alone or fused to HIF-1α residues 531-826 (Jiang et al. 1997) and empty vector or vector encoding HA-OS-9. For immunoblot assays, 293 cells were seeded at 3×10⁶ cells per 10-cm plate. The following day, the cells were co-transfected with expression vectors using Fugene-6. After 24 h, the cells were exposed to 20% or 1% O₂ with or without MG132 (10 μM) for 8 h and lysed in 200 μl of lysis buffer containing 50 mM Tris-Cl (pH 8.0), 150 mM NaCl, 5 mM EDTA, 0.1% SDS, 1% NP-40, 0.5% sodium deoxycholate, Complete protease inhibitor cocktail (Roche), sodium orthovanadate, and sodium vanadate (Sigma).
Immunoprecipitation Assays

293 cells (3×10⁶ cells per 100-cm plate) were transfected with Lipofectamine Plus and a total of 10 μg of expression vectors. After 24 h, cells were lysed with PBS/0.1% Tween-20. 400 μg of whole cell lysate (brought to a final volume of 200 μl with lysis buffer) were immunoprecipitated using anti-HA affinity matrix (Roche) followed by immunoblot assay using antibody against HIF-1α (Zhong et al. 1999), PHD 1-3 (Novus Biologicals Inc.), or HA (Roche).
Prolyl Hydroxylation Assay

[³⁵S]methionine-labeled VHL protein was synthesized in vitro and GST-HIF-1α (531-826) fusion protein (GstA) was expressed in E. coli. 293 cells were transfected with empty, PHD2, or OS-9 expression vector. After 24 h, cells were washed twice with cold hypotonic extraction buffer containing 20 mM Tris (pH 7.5), 5 mM KCl, 1.5 mM MgCl₂, 1 mM dithiothreitol and lysed with hypotonic extraction buffer in a Dounce homogenizer. The cell extract was centrifuged at 10,000×g for 10 min at 4° C., and the supernatant was stored in aliquots at −70° C. GstA (4 μg) was preincubated with whole cell lysate (50 μg) in a total volume of 50 μl with PBS-T at 30° C. for 10 min with or without 1 mM dimethyloxalyl glycine (DMOG). The reaction was terminated by addition of desferrioxamine to 1 mM. One aliquot (0.5 μg of GstA) was used for immunoblot assay using anti-hydroxyproline-564 HIF-1α antibody (Chan et al., 2002). The remainder (3.5 μg of GstA) was used for in vitro VHL binding assay.

VHL and PHD Binding Assays

For in vitro VHL binding assay, preincubated GstA and 5 μl of [³⁵S]methionine-labeled VHL protein were mixed in a total volume of 300 μl of PBS-T with 1 mM desferrioxamine and incubated at 4° C. for 90 min. 20 μl of glutathione-Sepharose-4B beads (Amersham Biosciences) was added and incubated for 30 min with rotation. Beads were washed three times with PBS-T. Proteins were eluted in Laemmli sample buffer, fractionated by SDS-PAGE, and detected by autoradiography. For PHD binding assay, 4 pg of GST-HIF-1α was preincubated with 50 μg of whole cell lysate in a total volume of 50 μl with PBS-T at 30° C. for 10 min with or without 1 mM DMOG. The reaction was terminated by addition of desferrioxamine to 1 mM, 20 μl of glutathione-Sepharose-4B beads were added and incubated for 30 min. The beads were washed three times with PBS-T. Bound protein was eluted in Laemmli sample buffer, fractionated by SDS-PAGE, and subjected to immunoblot assay using antibodies against PHD2 (Novus Biologicals Inc.) or GST (Amersham).

The mammalian expression vector, pSR.retro.GFP.Neo.circular.stuffer (OligoEngine) was used for expression of shRNA in 293 cells. The shRNA_{OS-9} insert consists of a 19-nucleotide sequence (gtacaaacagcgctatgag [SEQ ID NO:1]) corresponding to nucleotides 198-216 of OS-9 mRNA, which is separated by a spacer (ttcaagaga [SEQ ID NO:2]) from the reverse complement of the same 19-nucleotide sequence. A scrambled negative control vector (shRNA_{SNC}), constructed using a 19-nucleotide sequence (acgcatgcatgcttgcttt [SEQ ID NO:3]) with no significant homology to any mammalian gene sequence, served as a non-silencing control. Oligonucleotides were annealed and ligated into BglII- and HindIIIdigested vector. 293 cells were analyzed by fluorescence microscopy and lysed for RNA and protein isolation 24 h after transfection with shRNA expression vector.
RT-PCR and Real-time Assays

Total RNA was extracted from cells using RNeasy Mini Kit (Qiagen) and treated with DNase. Five μg of total RNA were used for first-strand synthesis with iScript cDNA Synthesis system (BioRad). cDNA was used for PCR analysis of OS-9, HIF-1α, and β-actin mRNA. Real-Time PCR was performed using iQ SYBR Green Supermix and the iCycler Real-Time PCR Detection System (BioRad). Expression of OS-9 mRNA relative to 18S rRNA was calculated based on the threshold cycle (C_{T}) as 2^{-Δ(ΔCT)}, where ΔCT=C_{T,target}−C_{T.}18s.
Statistical Analysis

Data are presented as mean ±SEM. Differences between experiments were analyzed for statistical significance (P<0.05) by ANOVA or two-sample t test.

### Example 1

Identification of OS-9 Interaction with HIF-1α

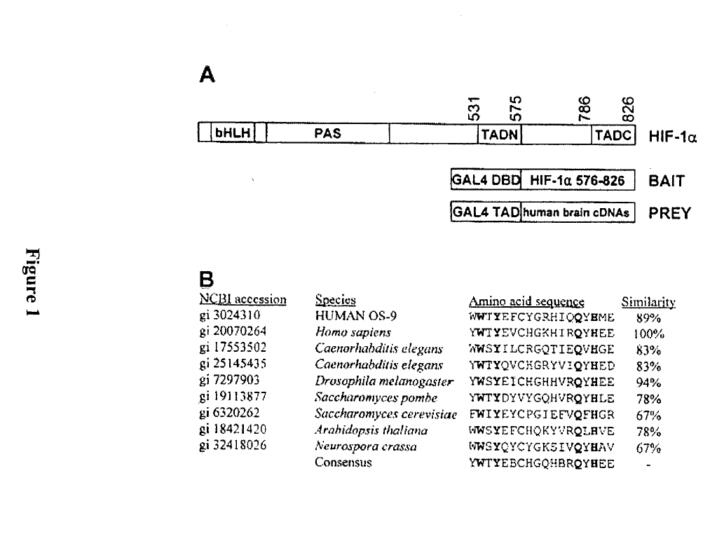

A yeast two-hybrid assay was performed to identify proteins that interact with the carboxyl terminus (amino acid residues 576-826) of HIF-1α. Yeast were transformed with a bait vector, which encoded a fusion protein consisting of the GAL4 DNA-binding domain and HIF-1α residues 576-826, and a prey vector, which contained human brain cDNA sequences fused to sequences encoding the GAL4 transactivation domain (FIG. 1 A). 2×10⁶ yeast transformants were subjected to a stringent series of positive and negative screens (see Materials and Methods) that resulted in the identification of 6 yeast colonies that exhibited both histidine auxotrophy and α-galactosidase (α-gal) activity. The nucleotide sequence of the human cDNA in the prey vector in each of these six yeast clones was determined. Three of the 6 clones contained prey vectors with cDNA that matched the sequence for human OS-9 (Unigene cluster Hs.76228, NCBI), an expressed sequence that was originally identified as amplified in osteosarcomas (Su et al. 1996) and is ubiquitously expressed (987 EST entries in the UniGene database). The prey vectors started at nucleotides 484 and 1106 of the full-length OS-9 mRNA sequence (GenBank accession number AB002806, NCBI) and encoded fusion proteins containing amino acids 49-667 and 357-667, respectively, of OS-9. Alternative splicing of the primary OS-9 RNA transcript results in the translation of 667-, 612-, and 597-amino-acid isoforms of OS-9 (Kimura et al. 1997, 1998). All 3 cDNAs contained partial sequences encoding the 667-residue isoform.

A HomoloGene database (NCBI) search revealed 617- and 693-amino-acid OS-9 homologues in mouse (NP_{—}808282) and rat (XP_{—}343219) with 70% and 73% identity, respectively, to human OS-9. BLAST analysis also identified three mouse ESTs (BI873098, BQ946493, and CD807089, NCBI) that encode a composite 667-amino-acid sequence with 78% identity to human OS-9. The 27.4-kb human OS9 gene (GeneID 109567, Entrez Gene database, NCBI) consists of 15 exons and is located on chromosome 12g13. Exons 1-6 encode the first 263 amino acids, which exhibit 94% identity with the mouse and rat OS-9 protein sequences. A search of the Conserved Domain Database (NCBI) revealed that this region of the OS-9 protein represents a domain of unknown function that is present in proteins from mammalian, invertebrate, yeast, and plant species. Within this extended domain, an 18-amino acid sequence (residues 117-134 of OS-9) shows particularly striking conservation (FIG. 1B).

### Example 2

Localization of HIF-1α/OS-9 Interaction

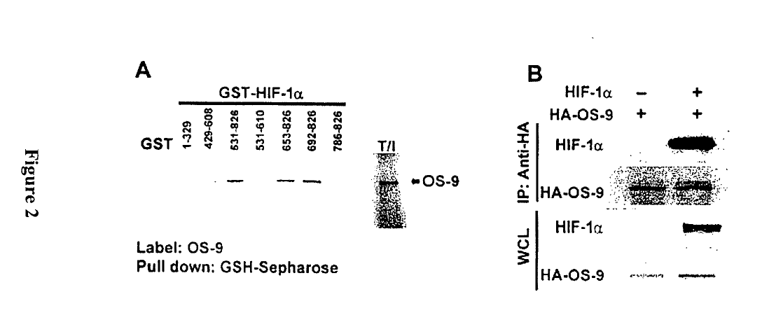

To demonstrate direct interaction between HIF-1α and OS-9 and to localize the HIF-1α residues required for interaction, bacterially expressed fusion proteins consisting of glutathione-S-transferase (GST) fused to HIF-1α sequences were incubated with ³⁵S-labelled in vitro-translated OS-9. The GST-HIF-1α proteins were recovered on glutathione-Sepharose beads and the binding of OS-9 was determined by gel analysis. HIF-1α residues 531-826, 653-826, or 692-826 efficiently bound to OS-9 whereas residues 1-329, 429-608, 531-610, and 786-826 did not bind OS-9 (FIG. 2A). These results indicate that HIF-1α residues 692-826 are sufficient and that residues 692-785 are necessary for binding to OS-9. To demonstrate that HIF-1α and OS-9 interact in living cells, human embryonic kidney 293 cells were co-transfected with expression vector encoding HA-tagged OS-9 and either empty vector or HIF-1α expression vector, and cell lysates were immunoprecipitated with anti-HA antibodies. Immunoblot assay revealed co-immunoprecipitation of HA-OS-9 and HIF-1α (FIG. 2B).

### Example 3

Modulation of HIF-1 Expression by OS-9

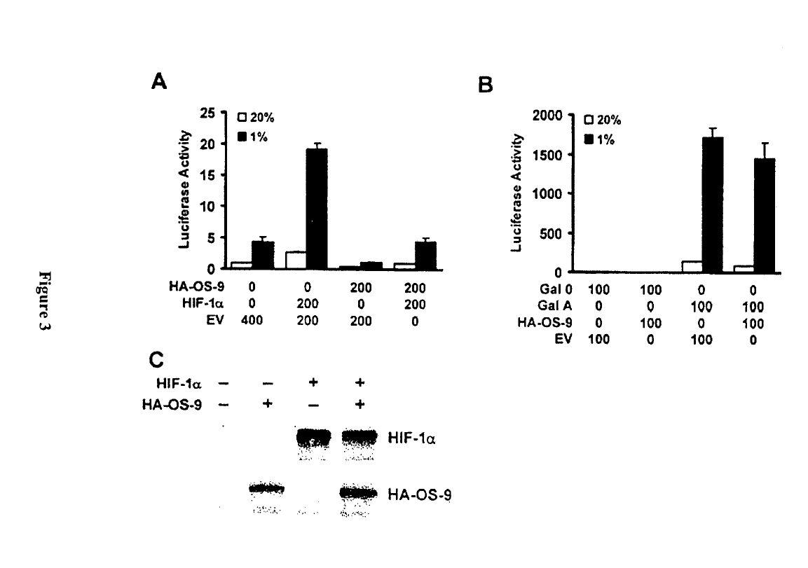

To investigate whether OS-9 modulates HIF-1 transcriptional activity, cells were co-transfected with HIF-1-dependent reporter plasmid p2.1, which contains a 68-bp hypoxia response element from the human ENO1 gene (Semenza et al. 1996), and expression vectors encoding HA-OS-9 and/or HIF-1α. Reporter gene activity that was induced by hypoxia and/or HIF-1α expression vector was dramatically inhibited by co-transfection of OS-9 expression vector (FIG. 3A). O₂ dependent hydroxylation events regulate HIF-1α protein stability and transactivation function. To determine whether OS-9 affects transactivation, 293 cells were co-transfected with a GAL4-dependent reporter, expression vector encoding either the GAL4 DNA-binding domain alone (pGalO) or fused to the HIF-1α transactivation domains (residues 531-826; pGalA), and empty vector or OS-9 expression vector. pGalA strongly transactivated the reporter in an O₂ regulated manner, as previously described (Jiang et al. 1997), and OS-9 had no significant inhibitory effect (FIG. 3B). To investigate whether OS-9 modulated HIF-1α protein levels, 293 cells were co-transfected with an expression vector encoding HIF-1α and either empty vector or OS-9 expression vector. In the presence of OS-9, HIP-1α protein levels were reduced (FIG. 3C).

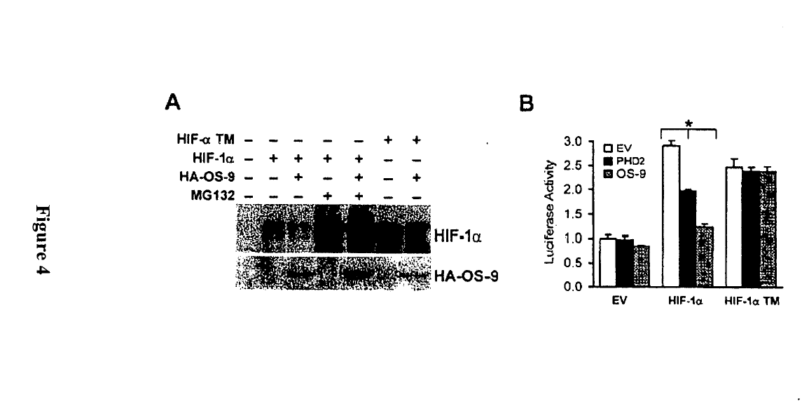

Decreased protein levels may be due to decreased production or increased destruction. The reduction in HIF-1α protein levels associated with OS-9 co-expression was blocked by treatment of cells with the proteasome inhibitor MG 132 (FIG. 4A). In addition, OS-9 had no effect on the levels of HIF-1α-TM, which contains a triple mutation of the hydroxylatable residues Pro-402, Pro-564, and Asn-803. These results suggested that the effect of OS-9 was mediated via the PHD-VHL-proteasome pathway. Therefore, the effect of overexpressing OS-9 or PHD2 were compared. PHD2 was chosen for analysis because of recent data indicating that this prolyl hydroxylase plays a predominant role in the regulation of HIF-1α expression (Berra et al. 2003). Induction of p2.1 reporter activity by HIF-1α was significantly inhibited by co-transfection of either OS-9 or PHD2 (FIG. 4B). In contrast, neither OS-9 nor PHD2 inhibited reporter gene transcription mediated by HIF-1 heterodimers containing HIF-1α-TM.

### Example 4

Affect of OS-9 on Prolyl Hydroxylation of HIF-1α

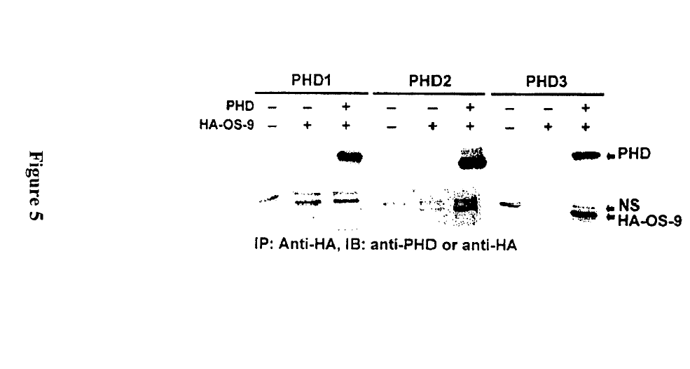

Hypothetically, OS-9 may function to increase the rate of prolyl hydroxylation by interacting with both HIF-1α and PHDs. To determine whether OS-9 also interacts with PHDs, co-immunoprecipitation assays were performed in cells expressing HA-OS-9 and either PHD 1, PHD2, or PHD3. Immunoblot analysis of anti-HA immunoprecipitates using antibodies that specifically recognize PHD1, PHD2, or PHD3 demonstrated interaction of OS-9 with each PHD (FIG. 5).

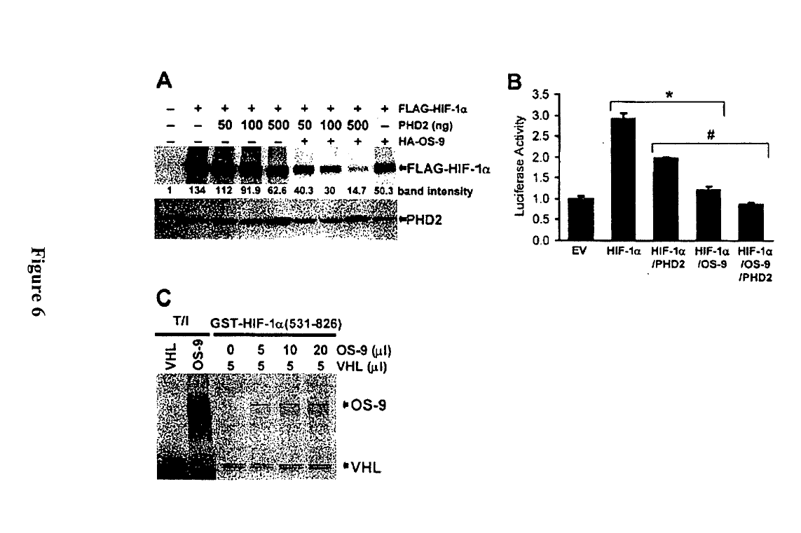

To provide further evidence that OS-9 promotes PHD activity, cells were co-transfected with expression vector encoding FLAG-tagged HIF-1α, and increasing amounts of PHD2 expression vector in the presence or absence of OS-9. The levels of HIF-1α were dramatically reduced in the presence of OS-9 (FIG. 6A, upper panel). This effect was not due to an increase in PHD2 protein levels (FIG. 6A, lower panel). Similar results were obtained when p2.1 reporter gene activity was assayed (FIG. 6B). Both studies also demonstrated that OS-9 had an inhibitory effect on HIF-1α protein levels and HIF-1transcriptional activity in the absence of co-transfected PHD2, which reflects functional interaction with endogenous PHDs. To rule out an effect of OS-9 on the binding of VHL to hydroxylated HIF-1α, GST-HIF-1α (531-826) was incubated with rabbit reticulocyte lysate as a source of prolyl hydroxylase activity, and then in vitro-translated OS-9 and VHL were added in the presence of desferrioxamine to block further hydroxylation. Although OS-9 bound to GST-HIF-1α (531-826) in a dose-dependent manner (FIG. 6C), increasing amounts of OS-9 had no effect on the binding of VHL to hydroxylated GST-HIF-1α (531-826).

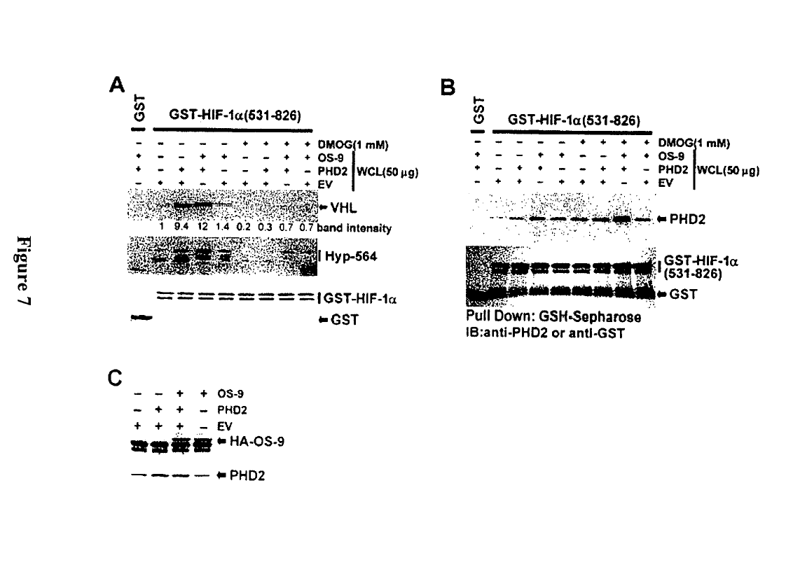

To directly demonstrate an effect of OS-9 on PHD-mediated hydroxylation of HIF-1α, in vitro hydroxylation of GST-HIF-1α (531-826) by PHD2 was assayed. 293 cells were transfected with expression vector encoding OS-9 or PHD2. Whole cell lysates were prepared and aliquots incubated with GST-HIF-1α (531-826), followed by addition of VHL. The binding of VHL was greatly increased following incubation of GST-HIF-1α (531-826) with lysates from PHD2-transfected cells (FIG. 7A, top panel). VHL binding was further increased when lysates from cells co-transfected with both PHD2 and OS-9 were used as a source of hydroxylase activity. In addition to O₂, the other substrate of the hydroxylation reaction mediated by PHDs is 2-oxoglutarate. Addition of dimethyloxalylglycine (DMOG), a competitive antagonist of 2-oxoglutarate, completely blocked binding of VHL to GST-HIF-1α (531-826). The increased VHL binding mediated by PHD2 or OS-9 was due to an increase in the prolyl hydroxylation of GST-HIF-1α (531-826) as determined by immunoblot assay using an antibody (Chan et al. 2002) that specifically recognizes HIF-1α containing hydroxyproline at residue 564 (FIG. 7A, middle panel). In vitro incubation of GST-HIF-1α (531-826) with lysates from cells transfected with both OS-9 and PHD2 vectors revealed increased binding of PHD2 to GST-HIF-1α (531-826) as compared to lysates transfected with PHD2 vector alone (FIG. 7B). Binding of PHD2 was further increased when prolyl hydroxylase activity was inhibited by DMOG. Co-transfection of OS-9 vector had no effect on PHD2 protein levels (FIG. 7C). Taken together the results in FIGS. 6 and 7 demonstrate that OS-9 stimulates prolyl hydroxylation of HIF-1α via the formation of a ternary complex with PHD2.

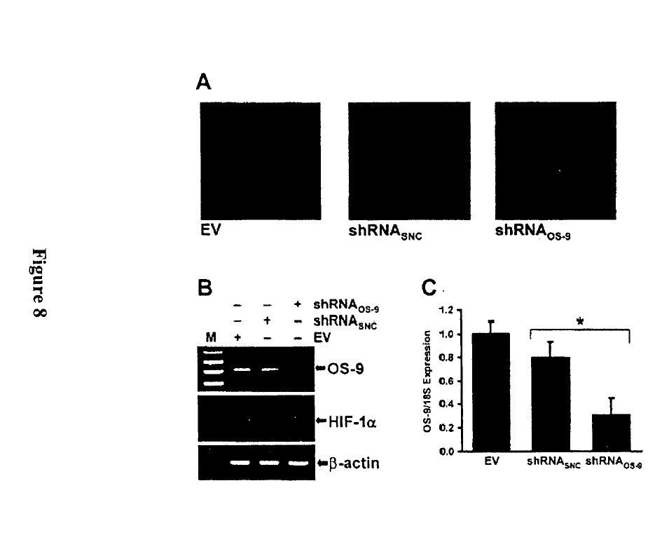

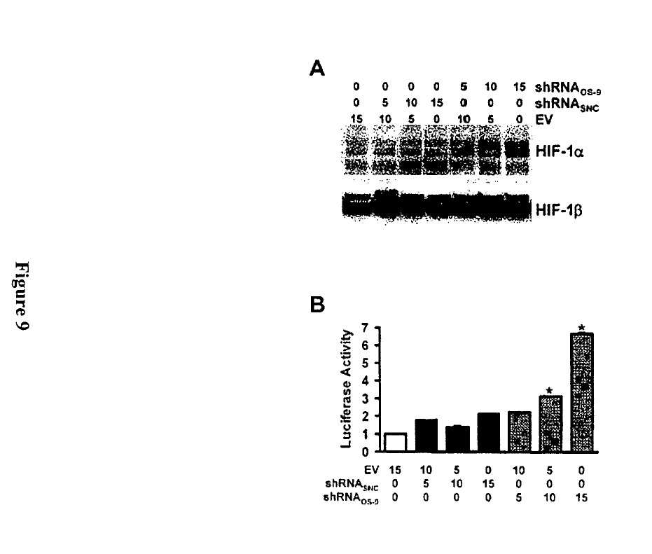

The above demonstrate that increased OS-9 expression results in decreased HIF-1α levels. To investigate the effect of OS-9 loss-of-function, 293 cells were transfected with an expression vector encoding green fluorescent protein (GFP) and a small hairpin RNA designed to target OS-9 mRNA for degradation (shRNA_{OS-9}). Cells were also transfected with empty vector or expression vector encoding a scrambled negative control shRNA (ShRNA_{SNC}). Fluorescence microscopy demonstrated similar transfection efficiency for each vector (FIG. 8A). Compared to cells expressing shRNA_{SNC}, OS-9 mRNA levels were significantly decreased in cells expressing shRNA_{OS-9}, as demonstrated by gel analysis of RTPCR products (FIG. 8B) and by real-time RT-PCR assays (FIG. 8C). A dose-dependent increase in HIF-1α protein levels (FIG. 9A) and HIF-1 transcriptional activity (FIG. 9B) was observed in cells expressing shRNA_{OS-9} but not in cells expressing shRNA_{SNC}. These results indicate that reduction of endogenous OS-9 levels is sufficient to increase HIF-1α levels under non-hypoxic conditions.

All of the compositions and methods disclosed and claimed herein can be made and executed without undue experimentation in light of the present disclosure. While the compositions and methods of this invention have been described in terms of illustrative embodiments, it will be apparent to those of skill in the art that variations may be applied to the composition, methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. Although the invention has been described with reference to the above examples, it will be understood that modifications and variations are encompassed within the spirit and scope of the invention.

What is claimed is:

1. A method of modulating hypoxia-inducible factor 1 (HIF-1) activity comprising:
a) contacting a sample comprising OS-9 and HIF-1 or a fragment thereof, with an agent that modulates OS-9 activity or expression; and
b) determining the effect of step (a) on the activity of HIF-1 or fragment thereof; wherein modulation of OS-9 activity or expression affects HIF-1 activity.

2. The method of claim 1, wherein the modulating agent inhibits the activity, synthesis, or stability of OS-9, resulting in increased HIF-1 activity or wherein the modulating agent stimulates the activity, synthesis, or stability of OS-9, resulting in decreased HIF-1 activity.

3. The method of claim 2, wherein the agent is an antibody, protein, small molecule, or a nucleic acid.

4. The method of claim 3, wherein the nucleic acid is an aptamer, antisense RNA, or gene silencing RNA.

5. The method of claim 4, wherein the gene silencing RNA is a dsRNA, siRNA, stRNA, or RNA silencing hairpin.

6. The method of claim 3, wherein the protein is an exogenous OS-9 isoform, which isoform exhibits activity antagonistic to the OS-9 endogenous to the sample.

7. The method of claim 6, wherein the sample is a cell, tissue, or organ transfected with an expression vector comprising an operably linked DNA encoding the exogenous isoform.

8. The method of claim 2, wherein increased HIF-1 activity stimulates angiogenesis, glucose metabolism, or cell survival.

9. The method of claim 2, wherein decreased HIF-1 activity inhibits angiogenesis, glucose metabolism, or cell survival.

10. The method of claim 1, wherein the determining step comprises analysis of OS-9 protein levels.

11. The method of claim 1, wherein OS-9 modulation affects interaction between OS-9 and HIF-1 and/or OS-9 and a prolyl hydroxylase (PHD).

12. The method of claim 11, wherein the interaction is determined by fluorescence resonance energy transfer (FRET) or two-hybrid assay.

13. The method of claim 1, wherein HIF-1 activity corresponds to HIF-1 protein stability and/or transactivation of O₂/hypoxia dependent gene expression via HIF-1.

14. The method of claim 13, wherein transactivation of O₂/hypoxia dependent gene expression can be monitored by determining expression of a gene, gene-fusion construct, or gene fragment, which gene, gene-fusion construct, or gene fragment expression is regulated by a hypoxia response element (HRE).

15. The method of claim 14, wherein the sample further comprises an HRE-containing expression vector, which expression from the vector is responsive to O₂/hypoxia dependent transactivation.

16. The method of claim 15, wherein the vector expresses a reporter protein.

17. The method of claim 16, wherein the reporter is luminescent.

18. The method of claim 17, wherein the vector expresses a fusion protein comprising HIF-1α, or a fragment thereof, and the gene reporter.

19. The method of claim 18, wherein the gene reporter is GFP, chloramphenicol acetyltransferase (CAT), β-galactosidase (β-Gal), alkaline phosphatase, or luciferase.

20. The method of claim 13, wherein HIF-1 protein stability can be monitored by determining interaction between HIF-1, an HIF-1 subunit, or an HIF-1 fragment and a PHD or PHD fragment, and/or a von Hippel-Lindau tumor suppressor protein (VHL), or VHL fragment.

21. The method of claim 20, wherein HIF-1 can be monitored by determining interaction between HIF-1, an HIF-1 subunit or HIF-1 fragment and FIH-1.

22. The method of claim 21, wherein the HIF-1 subunit is HIF-1α.

23. The method of claim 20, wherein the PHD is PHD1, PHD2, or PHD3.

24. The method of claim 13, wherein protein stability can be monitored by determining ubiquitylation of HIF-1, HIF-1α, or fragment thereof, which ubiquitylation results in degradation of HIF-1, HIF-1α, or fragment thereof by a proteasome.

25. The method of claim 13, wherein the sample is a cell, a tissue, or an organ and OS-9 dependent affects on HIF-1 protein stability and/or transactivation of O₂/hypoxia dependent gene expression effects modulation of glucose transporter expression, glycolytic enzyme expression, or growth/survival factor expression.

26. A method of identifying an OS-9 modulating agent comprising:
a) contacting a sample comprising OS-9 and HIF-1, an HIF-1 subunit, or a fragment thereof, with a test agent;
b) allowing interaction between the agent-contacted OS-9 and HIF-1, HIF-1 subunit, or a fragment thereof; and
c) determining HIF-1 activity,
wherein the test agent inhibits the activity, synthesis, or stability of OS-9, resulting in increased HIF-1 activity or wherein the test agent stimulates the activity, synthesis, or stability of OS-9, resulting in decreased HIF-1 activity.

27. The method of claim 26, further comprising determining the level of OS-9 protein subsequent to contacting with the test agent, wherein the sample is a cell, tissue, or organ.

28. An agent identified by the method of claim 26, wherein the agent is an RNA.

29. The agent of claim 28, wherein the RNA sequence is encoded by a nucleic acid comprising SEQ ID NO:1.

30. A pharmaceutical composition comprising a pharmaceutically acceptable carrier and a nucleic acid comprising SEQ ID NO:1.

31. The method of claim 26, wherein the agent is a small molecule, mineral, protein, peptide, hormone, nucleic acid, lipid, carbohydrate, vitamin, or co-enzyme.

32. The method of claim 26, further comprising determining HIF-1α protein levels, wherein the sample is a cell, tissue, or organ.

33. The method of claim 32, wherein the sample comprises an expression vector encoding a gene, gene-fusion construct, or gene fragment.

34. The method of claim 33, wherein expression from the vector is responsive to O₂/hypoxia dependent transactivation.

35. The method of claim 33, wherein the vector expresses a reporter protein.

36. The method of claim 35, wherein the reporter is luminescent.

37. The method of claim 36, wherein the gene reporter is GFP or luciferase.

38. The method of claim 36, wherein the vector expresses a fusion protein comprising HIF-1α, or a fragment thereof, and the gene reporter.

39. The method of claim 36, wherein the reporter comprises a gene-fusion construct regulated by a hypoxia response element (HRE).

40. The method of claim 39, wherein the gene-fusion construct comprises at least one HIF-1/OS-9 binding site.

41. The method of claim 26, wherein the test agent affects interaction between OS-9 and HIF-1, OS-9 and HIF-1α, or fragments thereof, and/or OS-9 and a prolyl hydroxylase (PHD).

42. The method of claim 41, wherein the interaction is determined by fluorsence resonance energy transfer (FRET) or two-hybrid assay.

43. The method of claim 41, further comprising determining the interaction between HIF-1, an HIF-1 subunit, or HIF-1 fragment and a PHD or PHD fragment, and/or a von Hippel-Lindau tumor suppressor protein (VHL), or VHL fragment.

44. The method of claim 43, wherein the PHD is PHD1, PHD2, or PHD3.

45. The method of claim 26, wherein determining is accomplished by measuring an increase or decrease in HIF-1 protein stability and/or transactivation of O₂/hypoxia dependent gene expression via HIF-1, which measuring in the presence and absence of the agent correlates with OS-9 modulation.

46. The method of claim 45, wherein protein stability can be monitored by determining ubiquitylation of HIF-1, HIF-1α, or fragment thereof, which ubiquitylation results in degradation of HIF-1, HIF-1α, or fragment thereof by a proteasome.

47. The method of claim 45, wherein the sample is a cell, tissue, or organ and OS-9 dependent affects on transactivation of O₂/hypoxia dependent gene expression effects modulation of glucose transporter expression, glycolytic enzyme expression, and growth/survival factor expression.

48. A method of modulating a regulator of O₂ homeostasis in a subject comprising altering the expression, stability, or activity of OS-9.

49. The method of claim 48, wherein the regulator is hypoxia inducible factor I (HIF-1).

50. The method of claim 49, further comprising administering to the subject or contacting the subject with an agent which modulates OS-9 expression, stability, or activity.

51. The method of claim 50, wherein the modulating agent is a small molecule, nucleic acid, or protein.

52. The method of claim 51, wherein the agent inhibits the activity, synthesis, or stability of OS-9, resulting in increased HIF-1 activity or wherein the agent stimulates the activity, synthesis, or stability of OS-9, resulting in decreased HIF-1 activity.

53. The method of claim 52, wherein OS-9 activity, expression, or stability is reduced by the modulating agent.

54. The method of claim 53, wherein the modulating agent is an antibody, aptamer, or nucleic acid.

55. The method of claim 54, wherein the nucleic acid is antisense RNA, dsRNA, siRNA, stRNA, or RNA silencing hairpin directed against OS-9 mRNA.

56. The method of claim 52, wherein the subject demonstrates an ischemic condition.

57. The method of claim 56, wherein the condition is a coronary, cerebral, or vascular disorder.

58. The method of claim 56, wherein the agent inhibits the activity, synthesis, or stability of OS-9, resulting in increased HIF-1 activity.

59. The method of claim 58, wherein increased HIF-1 activity stimulates angiogenesis, glucose metabolism, or cell survival.

60. The method of claim 59, wherein the agent inhibits the synthesis or stability of OS-9 protein or mRNA or the agent inhibits the interaction between OS-9 and HIF-1, HIF-1 subunit or fragment thereof, or the interaction between OS-9 and PHDs.

61. The method of claim 52, wherein OS-9 activity, expression, or stability is increased by the modulating agent.

62. The method of claim 61, wherein the agent is an OS-9 isoform, a small molecular weight compound or a vehicle encoding OS-9 or an OS-9 isoform.

63. The method of claim 62, wherein the vehicle is a plasmid or viral vector.

64. The method of claim 52, wherein the subject demonstrates a cell proliferating disorder.

65. The method of claim 64, wherein the disorder is cancer.

66. The method of claim 64, wherein the agent stimulates the activity, synthesis, or stability of OS-9, resulting in decreased HIF-1 activity.

67. The method of claim 66, wherein decreased HIF-1 activity inhibits angiogenesis, glucose metabolism, or cell survival.

68. The method of claim 67, wherein the agent stimulates the synthesis or stability of OS-9 protein or mRNA or the agent stimulates the interaction between OS-9 and HIF-1, HIF-1 subunit or fragment thereof, or the interaction between OS-9 and PHDs.

69. A method of treatment comprising administering to a subject in need thereof a pharmaceutically acceptable carrier comprising an OS-9 modulating agent, which agent alters the expression, stability, or activity of OS-9.

70. The method of claim 69, wherein the agent inhibits the activity, synthesis, or stability of OS-9, resulting in increased hypoxia inducible factor 1 (HIF-1) activity or wherein the agent stimulates the activity, synthesis, or stability of OS-9, resulting in decreased HIF-1 activity.

71. The method of claim 70, wherein OS-9 activity, expression, or stability is inhibited by the modulating agent.

72. The method of claim 71, wherein the inhibition of OS-9 results in increased HIF- l activity, which increased HIF-1 activity stimulates angiogenesis, glucose metabolism, or cell survival.

73. The method of claim 72, wherein the subject presents an ischemic condition.

74. The method of claim 73, wherein the condition is a coronary, cerebral, or vascular disorder.

75. The method of claim 70, wherein OS-9 activity, expression, or stability is increased by the modulating agent.

76. The method of claim 75, wherein the stimulation of OS-9 results in decreased HIF-1 activity, which decreased HIF-1 activity reduces angiogenesis, glucose metabolism, or cell survival.

77. The method of claim 75, wherein the subject presents a cell proliferating disorder.

78. The method of claim 77, wherein the disorder is cancer.


SEQUENCE LISTING (14 sequences)

SEQ ID NO: 1 - DNA, 19 bp, Artificial sequence
  Synthetic construct
gtacaaacagcgctatgag

SEQ ID NO: 2 - DNA, 9 bp, Artificial sequence
  Synthetic construct
ttcaagaga

SEQ ID NO: 3 - DNA, 19 bp, Artificial sequence
  Synthetic construct
acgcatgcatgcttgcttt

SEQ ID NO: 4 - protein, 667 aa, Homo sapiens
MAAETLLSSLLGLLLLGLLLPASLTGGVGSLNLEELSEMRYGIEILPLPVMGGQSQSSDVVIVSSKYKQRYECRLPAGAIHFQREREEETPAYQGPGIPELLSPMRDAPCLLKTKDWWTYEFCYGRHIQQYHMEDSEIKGEVLYLGYYQSAFDWDDETAKASKQHRLKRYHSQTYGNGSKCDLNGRPREAEVRFLCDEGAGISGDYIDRVDEPLSCSYVLTIRTPRLCPHPLLRPPPSAAPQAILCHPSLQPEEYMAYVQRQADSKQYGDKIIEELQDLGPQVWSETKSGVAPQKMAGASPTKDDSKDSDFWKMLNEPEDQAPGGEEVPAEEQDPSPEAADSASGAPNDFQNNVQVKVIRSPADLIRFIEELKGGTKKGKPNIGQEQPVDDAAEVPQREPEKERGDPERQREMEEEEDEDEDEDEDEDERQLLGEFEKELEGILLPSDRDRLRSEVKAGMERELENIIQETEKELDPDGLKKESERDRAMLALTSTLNKLIKRLEEKQSPELVKKHKKKRVVPKKPPPSPQPTEEDPEHRVRVRVTKLRLGGPNQDLTVLEMKRENPQLKQIEGLVKELLEREGLTAAGKIEIKIVRPWAEGTEEGARWLTDEDTRNLKEIFFNILVPGAEEAQKERQRQKELESNYRRVWGSPGGEGTGDLDEFDF

SEQ ID NO: 5 - protein, 18 aa, Homo sapiens
WWTYEFCYGRHIQQYHME

SEQ ID NO: 6 - protein, 18 aa, Homo sapiens
YWTYEVCHGKHIRQYHEE

SEQ ID NO: 7 - protein, 18 aa, Caenorhabditis elegans
WWSYILCRGQTIEQVHGE

SEQ ID NO: 8 - protein, 18 aa, Caenorhabditis elegans
YWTYQVCHGRYVIQYHED

SEQ ID NO: 9 - protein, 18 aa, Drosophila melanogaster
YWSYEICHGHHVRQYHEE

SEQ ID NO: 10 - protein, 18 aa, Saccharomyces pombe
YWTYDYVYGQHVRQYHLE

SEQ ID NO: 11 - protein, 18 aa, Saccharomyces cerevisiae
FWIYEYCPGIEFVQFHGR

SEQ ID NO: 12 - protein, 18 aa, Arabidopsis thaliana
WWSYEFCHQKYVRQLHVE

SEQ ID NO: 13 - protein, 18 aa, Neurospora crassa
WWSYQYCYGKSIVQYHAV

SEQ ID NO: 14 - protein, 18 aa, Artificial sequence
  Consensus sequence
YWTYEBCHGQHBRQYHEE